(19) United States
(12) Patent Application Publication
(10) Pub. No.: US 2026/0199469 A1
(43) Pub. Date: Jul. 16, 2026

(54) BIOSPECIFIC IN TANDEM RECEPTOR CAR AND METHOD FOR MODULATING THE TUMORAL MICROENVIRONMENT

(71) Applicant: CELLURIS LLC, LEWES, DE (US)

(72) Inventor: Patricia Bortman Rozenchan, Sao Paulo (BR)

(21) Appl. No.: 19/343,731

(22) Filed: Sep. 29, 2025

Related U.S. Application Data
(63) Continuation of application No. 17/420,739, filed on Jul. 6, 2021, now Pat. No. 12,491,247.

(51) Int. Cl. A61K 40/41; A61K 39/00; A61K 40/11; A61K 40/31; A61K 40/42; C07K 14/725; C07K 16/28; C07K 16/30

(52) U.S. Cl. CPC A61K 40/41; A61K 39/0011; A61K 40/11; A61K 40/31; A61K 40/421; A61K 40/4211; A61K 40/4215; A61K 40/4217; A61K 40/4255; C07K 14/7051; C07K 16/2803; C07K 16/2866; C07K 16/2878; C07K 16/30; A61K 2239/29; C07K 2317/31; C07K 2317/622; C07K 2319/30

(57) ABSTRACT

The present invention refers to a bispecific in tandem receptor CAR, named RfuCAR, which includes a scFv that recognizes and ligates surface molecules on tumoral cells (CD33, CD123 or another tumoral target) and the IL-1 receptor type 2 (IL-1R2). According to this, the IL1-R2 was chosen as the ideal receptor to compose the RfuCAR construction, being able to capture the IL-1B with high affinity and specificity. These proprieties indicate it as a good candidate to reduce the neurotoxicity and CRS effects of CAR-T therapies. Additionally, the present invention deals with a method for modulating the tumoral microenvironment, for example, in case of acute myeloid leukemia, or other cancer type like but not restricted to acute lymbloblastic leukemia, pancreatic, lung and ovarian cancer.

### FIELD OF THE INVENTION

The present invention belongs to the field of Medicine, more precisely in the area of Immunology, cell, biology and molecular biology, and describes a bispecific in tandem receptor CAR, named RfuCAR, which includes a scFv that recognizes and ligates surface molecules on tumoral cells (CD33, CD123 or another tumoral target, like, but not restricted to CD19, Mesothelin and BCMA, and the IL-1 receptor type 2 (IL-1R2), as well as a method for modulating the tumoral microenvironment, for example, in case of acute myeloid leukemia, or other cancer type, like but not restricted to acute lymphoblastic leukemia, pancreatic, lung and ovarian cancers.

### BACKGROUND OF THE INVENTION

### Interleukin 1 Receptor (IL-1R)

The interleukin-1 (IL-1α and IL-1β) is a prototypic multifunctional cytokine which, differently from other cytokines, is able to affect nearly every cell type concurrently or not with other cytokines or small mediator molecules (DINARELLO, 1996). This cytokine has pleiotropic effects as an immune and inflammatory mediator (DINARELLO, 1996; APTE et al., 2002).

The main members of IL-1 family consist of IL-1α, IL-1β, IL-1 receptor antagonist (IL-1ra), IL-1 receptor type I (IL-1RI), IL-1 receptor type II (IL-1RII) and IL-1 receptor accessory protein (IL-1RAcP) (DINARELLO, 1996; DUNN et al., 2001). Other cytokines named IL-18, IL-1F5, IL-1F6, IL-1F7, IL-1F9 and other are also included in the IL-1 family (DUNN et al., 2001; DINARELLO, et al., 2010).

IL-1 family are cytokines that control the pro-inflammatory reactions in response to pathogen-associated tissue injury and damaged and/or danger-associated molecular patterns; therefore, are the major mediators of innate immune reactions (WEBER, et al., 2010).

IL-1 signaling and expression are tightly regulated events comprising of gene expression controlling, synthesis and secretion and also control of surface receptors, soluble receptors and receptor antagonists (DINARELLO, 1996). This cytokine has innumerous effects including fever, increased hepatic acute phase response, increased metastases, angiogenesis, increased antibody and lymphokine production, cartilage breakdown, proliferation of fibroblasts, smooth muscle and mesangial cells and increased HIV-1 gene expression (AURON,1998).

IL-1 mediates the increase in endothelial adhesion molecules that facilitates the emigration of neutrophils into the tissues but also to the metastatic niche (VIDAL-VANACLOCHA, et al. 1996). This cytokine is also involved in the angiogenesis and vascular endothelial growth factor (VEGF) production (COXON, et al., 2002; VORONOV, et al. 2003; SONG, et al., 2003). The levels of all IL-1 family members are increased in acute myeloid leukemia (AML) patients, leading to a significant suppression of normal progenitors clonogenicity and disease progression (CAREY, et. al. 2017). In spite of the high sequence similarity between IL-1α and IL-1β (DUNN et al., 2001), there are remarkably differences in its biological actions: IL-1α reduces tumorigenicity by inducing antitumor immunity whereas IL-1β promotes invasiveness including tumor angiogenesis and immune suppression in the host (SONG, et al., 2003). This pro-tumorigenic effects of IL-1β were observed in different cancer models such as melanoma, mammary and prostate cancers and also tumoral cells and macrophages cultures (VORONOV, et al. 2003).

The deregulated or excessive activation of IL-1 receptors is the potential cause of dangerous and detrimental local or systemic inflammatory reactions, as well as autoimmune or allergic responses (BONECCHI, et al. 2016). Recently the role of IL-6 and IL-1 in the CRS and neurotoxicity induced by CAR-T therapy was demonstrated, indicating that IL-1 is a better target in order to control both of those adverse events (NORELLI, et al. 2018; GIAVRIDIS, et al. 2018; TARASEVICIUTE, et al. 2018). The blocking of IL-1 expression in patients with myeloma reduces IL-6 production and extends progression-free survival (LUST, et al. 2009). The IL-1 receptor antagonist (IL1-RA) has been applied to treat countless diseases, such as rheumatoid arthritis, asthma, septic shock, graft versus host disease, Alzheimer's disease, arteriosclerosis, multiple myeloma and adult T-cell leukemia (HALLEGUA, 2002). There is enough evidence to support IL-1 blockage as a good approach to treat human metastatic diseases (DINARELLO, et al. 1991).

### IL-1 Receptors

There are two main receptors for IL-1, type I and II that are located in the long arm of chromosome two and encodes for a 552 amino acids long and 80 kDa and a 336 amino acids long and 68 kDa, respectively (DINARELLO, 1996; DINARELLO, et al. 1991). These two receptors are members of the immunoglobulin superfamily, each are composed of three IgG-like domains and share significant homology to each other (DINARELLO, 1996). The type 1 receptor is found primarily on T cells, endothelial cells, keratinocytes, hepatocytes and fibroblasts, whereas the type 2 receptor is found on neutrophils, B cells and bone marrow cells. However, it is likely that some cells can express both types (DINARELLO, et al. 1991).

The IL-1R1 and IL-1R2 have different affinities for the three main ligands of IL-1 family (IL-1α, IL-1β and IL-1RA). IL-1R1 binds IL-1α with higher affinity and in opposite IL-1R2 binds IL-1β with higher affinity. Also, IL-1R2 binds IL-1RA 100 less efficiently than IL-1R1 (MANTOVANI, et. al. 1998; COLOTTA, et al. 1993). IL-1R2 shares 28% aminoacid homology with the extracellular portion of IL-1R1 but differs for the absence of a TIR domain and for the presence of only a short 29 amino acid-long cytoplasmic tail (BONECCHI, et al. 2016; MCMAHAN, et al. 1991). The IL-1R2 is unable to transduce signal being a decoy receptor (AURON, 1998; THOMAS, et al. 2014). IL-1 acts on myelomonocytic cells through IL-1R1 and IL-1R2 inhibits this cytokine activity acting as a trap for IL-1 agonists (COLOTTA, et al. 1993). Type 2 soluble IL-1R inhibits IL-1β at two steps, by preventing processing of propeptide and by blocking the interaction of mature IL-1β with type 1 IL-1 receptor (SYMONS, et al. 1995; BOURKE, et al. 1995).

The IL-1R2 is increased in the presence of glucocorticoid hormones (e.g. dexamethasone), prostaglandins, aspirin, Th2 cytokines, IL-10 and IL-27 contributing to immunosuppressive and anti-inflammatory activities (BONECCHI, et al. 2016; R E, et al. 1994). The anti-inflammatory role of IL-1R2 was demonstrated in several diseases including chronic skin inflammation (RAUSCHMAYR, et al. 1997), arthritis (BESSIS, et al. 2000; DAWSON, et al. 1999; ATTUR, et al. 2000), endometriosis (KHOUFACHE, et al. 2012; BELLEHUMEUR, et al. 2005; GUAY, et al. 2007), heart transplantation (SIMEONI, et al. 2007) and autoimmune myocarditis by Th17 cells (CHANG, et al. 2013).

### Spacers and Linkers

The CAR-mediated T-cell recognition is defined by the antibody domain and is independent of MHC presentation. This recognition is extended to any target in which the antibody is available (CHMIELEWSKI, et al. 2013). The interactions are strongly influenced by the structure and density of the target molecule on the tumor and the location of the epitopes that is recognized indicating that the sequence between the scFv and the T-cell membrane is crucial and should provide flexibility (HUDECEK, et at. 2015). The length of spacer may vary according to the target molecule and this logical thinking can also be applied for the region between two recognition sites in bi-specific CARs (HUDECEK, et at. 2015; GRADA, et al. 2013; HEGDE, et al. 2016). Also, the spacer characteristics are key in the modulation of transgenic cell phenotype, activation status, migratory capacity, and tumor recognition affecting the CAR potency (WATANABE, et al. 2016; NORELLI, et al. 2016).

Many combinations of spacer have been tested on literature and the suitability of each one is directly related to the disease to be treated and the targets chosen (CHMIELEWSKI, et al. 2013). GRADA, et al. demonstrated that for CD19 and Her2 tandem CAR, the Her2-scFv must be put in a just a-membrane position and the CD19-scFv in the distal position to allow for more relaxed and potentially simultaneous binding (GRADA, et al. 2013). A CAR that targets carcinoembryonic antigen (CEA) has the same behavior as CD19-scFv demonstrating a higher degree of T-cell activation when targets epitopes closer to the cell membrane (HOMBACH, et al. 2000). This is also reported for CARs targeting other epitopes (GUEST, et al. 2005; JAMES, et al. 2008).

These findings corroborate the kinetic-segregation model that suggests that targeting membrane distal epitopes increase the CAR-ligand clusters, which permits a more intense entering of phosphatases molecules in the synapse and repression of TCR signaling than proximal epitopes (DAVIS, et al. 2006). However, this model does not exclude the need for accessible and flexible epitopes, indicating that the best suitable target epitope and binding affinity for optimal CAR T-cell activation remains so far to be empirically evaluated in each case (CHMIELEWSKI, et al. 2013).

The specific requirements of the spacers or non-antigen binding components of the CAR in the extracellular domain must be carefully chosen and proven in vitro and in vivo (HUDECEK, et at. 2015). Some differences can be observed in the most characterized CD19-CAR as the antitumor efficacy is greater when a short spacer sequence derived from CD8a links the scFv to the intracellular signaling domains (PORTER, et al. 2011; KALOS, et al. 2011) than a IgG1 hinge and Fc (HUDECEK, et at. 2015; SAVOLDO, et al. 2011) or without a spacer (REN-HEIDENREICH, et al. 2000; MORITZ, et al. 1995). Patel (PATEL, et al. 1999) reported that CARs targeting HIVₑₙᵥ containing CD7 or IgG1-derived spacers demonstrated optimal cell lysis compared to CARs containing CD8, truncated CD4 or truncated IgG1-derived spacer; reinforcing that the spacer choice must be disease and target specific.

The previous works indicate that even if the spacer domain could provide flexibility for the extracellular domain increasing the distance from membrane, or from other antigen-binding domain, it can impair the T-cell activation, demonstrating that the improvement of binding will not necessarily result in increased CAR signaling (HOMBACH, et al. 2000; HAWKINS 2014). The controversial data in the literature, strongly varying between different targeted CARs indicates the requirement of in vitro and in vivo putative constructions testing.

### Linkers, Spacers and Hinges

### Gly-Ser Linker

Grada (GRADA, et al e 2013) constructed a tandem CAR for CD19 and Her2. The spacer/linker between the two recognition motifs were a sequence of glycin and serine aminoacids. Tandem repeats occur in at least 14% of the proteins with less than 2,000 residues and do not form the standard secondary structures, such as a-helix or B-sheets (MATSUSHIMA, et al. 2008). Indicating that Gly-Ser tandem repeats are highly flexible and non-cleavable allowing for near-free motion of the CAR subunits (MATSUSHIMA, et al. 2008). The tandem repeats seem to allow a structural flexibility that enables the interaction with various ligands including metal ions and other proteins (MATSUSHIMA, et al. 2008).

### IgG1

The IgG1 is the most abundant immunoglobulin class and for instance is widely applied as spacer in CAR constructions (VIDARSSON, et al. 2014). The spacer can comprise the whole IgG1 Fc domain (CH₂CH₃), Fc domain and hinge or only the hinge. The hinge comprises a 15 aminoacids between CH₁ and CH₂ domains (VIDARSSON, et al. 2014). Guest et al demonstrated that the IgG1 Fc domain space is not necessary for CD19 CAR optimal function (GUEST, et al. 2005) and can abrogate efficacy of CD19 CAR-T cell in mice (ALMASBAK, et al. 2015). Also, Hombach (HOMBACH, et al 2000) demonstrated that extracellular IgG1 domains impairs antigen dependent cellular activation in an anti-CD30 model. On the other hand, several authors have efficient CAR constructions with IgG1 Fc domains, i.e. CAR-PSCA and CAR-MUC1 (ANURATHAPAN, et al. 2014), NGFR-spaced-CD44v6, NGFR-spaced-CD19 and NGFR-spaced-CEA (CASUCCI, et al. 2015; CASUCCI, et al. 2018), CAR-PSA (WATANABE, et al. 2016). Moritz (MORITZ, et al 1995) have tested different constructions using hinge domains between function CAR regions. One of the concerns of IgG1 Fc application is that this domain is responsible for the complement cascade activation and it is enhanced when the antibody Fab domain is deleted (WANG, et al. 2016). In spite of putative effects of Fc chain in immune activation, experimental results using IgG1 indicates it can be a good spacer candidate, but it's effects probably will be only detected in the experimental assays.

### IgG4

The IgG4 class is less abundant than IgG1, having a very similar structure differing on the hinge domain size (12aa) (VIDARSSON, et al. 2014). The IgG4 antibodies are often formed following repeated or long-term exposure to antigen in a non-infectious setting (VIDARSSON, et al. 2014). IgG4 is functionally monovalent, which indicates it is less suitable to undesirable crosslinks (AALBERSE, et al. 2002). Probably due these characteristics IgG4 Fc and hinge were already being applied in CAR constructions. Generally, the IgG4 sequence is applied with hinge and Fc CH₂CH₃ domains (QIN, et al. 2017) or composed with sequences from other IgGs. The replacement of the first six amino acids of the CH₂ domain of IgG4 (APEFLG) with the corresponding five amino acids of IgG2 (APPVA) abrogates binding to Fc receptor and is necessary for tumor recognition in vivo (HUDECEK, et al. 2015). The need for alterations on IgG4 CH₂ region were reported by other authors, which mutated the CH₂ region in two sites (L235E; N297Q) and incorporate a deletion (CH₂ residues 235 and 297) leading to reduction on Fc receptor binding without altering the ability of the CAR to mediate antigen-specific lysis (JONNALAGADDA, et al. 2015). Deletions and mutations on IgG4 CH₂ portion abolish cell cytotoxicity and strongly reduced the complement activation by IgG4 (MONTANO, et al. 2002; DORAI, et al. 1992).

### Other Spacers

The CD4, CD8 and CD28 trans-membrane and hinge are widely applied in CAR constructions (NORELLI, et al. 2016). In a CD19 CAR, the hinge and trans-membrane regions from either CD8α or CD28 have similar function in mice, but CD8α seems to have lower levels of inflammatory cytokine production and activation-induced cell death (REN-HEIDENREICH, et al. 2000; ALABANZA, et al. 2017). These motifs usually are applied as spacers between scFv and T-cell membrane in monospecific CARs.

### Acute Myeloid Leukemia CAR Tested Spacers

According to the information discussed above, we choose the IgG4 Fc domain and hinge, including its variations (Hinge only, Hinge-CH₂CH₃ and CH₂CH₃ only) to apply as a spacer between the scFv receptor and IL1-R2 receptor in the RfuCAR construction. As shown previously this spacer is less reactive and seems to not interfere in the CAR regular function (JONNALAGADDA, et al. 2015; MONTANO, et al. 2002; DORAI, et al. 1992; JENA, et al. 2010). Also, is important to relate the spacer to the targeted disease of RfuCAR, so, for other tumoral targets we may change this option by any other spacer that presents better suitability. The first disease to be treated will be acute myeloid leukemia (AML) with the anti-CD33 and anti-CD123 CARs. On that regard, the use of IgG4 Fc and hinge has been also tested in other constructions. Many authors have successfully used the IgG4 Fc and hinge as spacer in CD33 (KENDERIAN, et al. 2015), CD123 (MARDIROS, et al. 2015; THOKALA, et al. 2016) and other targets (LABORDA, et al. 2017) CAR-Ts against AML (KENDERIAN, et al. 2017) (CD33 and CD123 Novartis Patents).

### PRIOR ARTS

It is noticed that the use of genetically modified chimeric T-cell receptor antigens expressing CD33 and/or CD123 antigens for the treatment of cancer has been of considerable interest to the scientific community in recent years. For example:

The document US 2013/280220 A1 refers to computational modeling tools to guide the design and construction of a novel single CAR molecule (TanCAR), recognizing each target molecule individually, capable of mediating the bispecific activation and targeting of T cells. Accordingly, with the fourth paragraph of this document: the present invention is directed to methods and compositions related to cell therapy. In particular embodiments the cell therapy is for cancer including solid tumors. However, the inventors use Glycine, Serine or both as linkers. The construct of the present invention uses IgG4 Fc domain as a spacer between the scFv receptor and IL1-R2 receptor and CD8 hinge as spacer between scFv and T-cell membrane in the RfuCAR construction, in addition to have different targets.

The document WO 2014/186469 A2 relates to methods and compositions for immunotherapy employing a modified T cell comprising a clinical grade antigen chimeric (CAR) receptor, which can be directly administered to the cancer treatment. With those modifications the invention will now have the ability to recycle effector functions within the tumor microenvironment. However, the inventors used transposon system to transduce T cells, we are using lentiviral vectors. Besides, they did not construct CARs using two different receptors at the same time for different targets, instead, they fused a mutein IL15 with a CD19 receptor in the construct.

The document WO 2014/055442 A2 relates to compositions and methods for the treatment of human cancer. The methodology of this document directs cells to a tumor microenvironment and includes a Chimeric Antigen Receptor (CAR) comprising an antigen binding domain, a transmembrane domain, a costimulatory signaling region and a zeta CD3 signaling domain, wherein the antigen-binding domain binds to a stromal cell antigen. Although the inventors for this patent want to affect the tumor microenvironment, they try to achieve this in a different manner than the RfuCAR of the present invention. While we want to reduce the IL-1 present in the microenvironment, the present invention comprises of an antigen-binding domain that binds to a stromal cell antigen, for example FAP (fibroblast activation protein).

The document WO 2017/222593 A1 relates to compositions and methods related to chimeric antigen receptors. More precisely, this document relates to genetically modified cells having the chimeric antigen receptors directed to at least two targets, for example, CD33 and CD123, among numerous others. However, such document presents a different way to construct the CAR, in the same CAR construct they insert two antigen recognition sites, also, they put a fused protein in the construct to serve as an enhancer. Our CAR presents two different receptors linked among each other. Their switch off mechanism is achieved through a suicide gene, RfuCAR has an on/off switch as well as the possibility of fine tuning the responses.

The document U.S. Pat. No. 9,815,901 B2 relates to the treatment of diseases related to CD123 expression. To this end, the document relates to a chimeric antigen receptor specific for CD123, in addition to proposing methods for the administration of genetically modified cells expressing the CD123 binding domain. Such document differs from the present invention in that it does not suggest the construction of chemical receptors, only mentioning it as being the recognition of CD123 antigens for the treatment of diseases related to the expression of such antigen. Additionally, this construct uses Glycine/Serine as linker. Also, they constructed a CAR which pursue an antigen recognition site for CD123 and CD19. The activation of this CAR occurs just when two antigens were linked by receptors. In order to control toxicity, the authors propose a dimerization switch, in which the signal transduction produced by the CART recognition will only be transmitted in the presence of a molecule that will perform a dimerization of the two CART parts. In the present invention, it is proposed a regulation of the immune microenvironment through the sequestration of IL-1 from the tumor microenvironment, and the possibility to manage the toxicity through the administration of peptides directed to the receptors described.

The document WO 2017/173256 A1 relates to compositions and methods comprising genetically modified immune cells expressing antigen (CAR) receptors or T-cell receptors (CAR-T) which are specifically directed to kill cancer cells. Although this document mentions immunotherapy by CAR-T, it is not specific for the recognition of the CD33 and CD123 antigens, also does not target the construction of chimeric receptors using such genetically modified cells, as in the present invention. In addition, the CAR construct comprised in this document consists of a single antigen site and a truncated hinge domain. The targets used in such document are different from that of the present invention.

The document WO 2015/164594 A1 is related to chimeric antigen receptors directed to cells expressing an antigen. This document refers to therapy with modified T cells, although these are not necessarily for the recognition of CD123 or CD33 antigens. Additionally, such document is related to a CART-cell targeting the EGFR antigen. In addition, they insert a second transgene, a sequence for IL15/IL15Ra fusion protein expression. The construction was made for work through the density of the target.

The document entitled “Engineering chimeric antigen receptor-t cells for cancer treatment” makes an analysis on therapy with chimeric antigen receptor (CAR) cells that has been successfully applied in the treatment of B cell malignancies, highlighting its great potential in antitumor therapy. CAR-T cells can be designed to kill malignant cells specifically or remodel the tumor microenvironment by releasing soluble factors that regulate the function of stromal cells or immune cells, providing a powerful tool for targeting multiple components of the tumor ecosystem. Such document is a literature's review for CART-cell technology. It describes the concepts of tumor ecosystem, the distinct cancer-immune phenotype and the T-cell exhaustion mechanisms in immune evasion. It's also reviewed the functional challenges of CART-cell technology. At any moment it is cited the immune regulation mechanism that are proposed in the present invention.

The document entitled “Perspectives on chimeric antigen receptor t-cell immunotherapy for solid tumors” relates to specific chimeric antigen receptors CARs, which can cause robust activation of T cells to initiate the killing of the target tumor cells. This document describes some recent approaches and innovations for the genetic re-engineering of CARs T cells to combat the inhibitory influences found in the tumor microenvironment. In addition, they mention the difficulties on treating solid tumors and that tumor microenvironment could help with this matter. So, this publication supports the present invention.

The document entitled “T cells expressing CD123-specific chimeric antigen receptors exhibit specific cytolytic effector functions and antitumor effects against human acute myeloid leukemia” relates to chimeric antigens receptors specific for CD123 expressed on T cells, exhibiting specific cytolytic effector functions and antitumor effects against acute myeloid leukemia. As mentioned, the receptor suggested in this document is specific for the recognition of the CD123 antigen. However, such document is an experimental article where they designed two distinct epitopes for CD123 to be used in the constructions. Although the CART design of the article is similar to ours, same IgG4 Fc receptor hinge, CD28 used as co-stimulatory and CD3 zeta as signaling transducer, our CART design also contemplates the use of a second co-stimulatory molecule, 4-1BB. Another major difference of our CART is the immune microenvironment regulation mechanism that we propose; the paper's CART design does not contemplate any mechanism related to that function.

The document entitled “Switching CAR t cells on and off: a novel modular platform for retargeting of t cells to AML blasts” demonstrates that CD33 and CD123 are expressed either alone or in combination in patients with acute myeloid leukemia (AML) and thereafter a treatment against AML with chimeric T cell antigen receptors expressing both CD33 and CD123 was suggested. This document is very similar with the present invention since it suggests the use of genetically modified T cells which recognize the CD123 and/or CD33 antigens for the treatment of acute myeloid leukemia. Such paper shows a modular CAR construct, with dual targeting modules against the tumoral antigen, that can be switched on/off by its proper kind of construction. The construction of the present invention is made using two very different receptors, one for the tumoral antigen and the other one for IL-IRA, specifically.

In view of the above, it is concluded that the construct (RfuCAR) proposed in the present application is different from any other constructs because it comprises a mechanism that is able to modulate the tumoral microenvironment and also be switched-off by the administration of both receptor epitope peptides, constituting a regulatory safety switch, using as receptors the IL-1R2, which was not used for this end yet, besides the tumoral receptor target. If any of the toxic CAR-T effects are detected in the patient, the RfuCAR cells action can be transiently switched-off and also tuned by the administration of different peptides that link to IL-1R2, IL-1-IL-1R2 and/or scFv epitopes. These peptides will be able to link the two RfuCAR preventing the ligation to tumoral cells and/or IL-1 inhibiting or modulating RfuCAR activity. The RfuCAR cells will still be able to proliferate in the patient and get back to kill tumoral cells as soon as the administration of the peptides ceases. Accordingly, the construct and mechanism of the present invention are a novel approaches that aims at regulating the tumoral microenvironment not only through the cell signaling pathway, but also to the secreted molecules from macrophages and other cells.

### SUMMARY OF THE INVENTION

The present invention has the purpose to propose a bispecific in tandem receptor CAR, named RfuCAR, which includes a scFv that recognizes and ligates surface molecules on tumoral cells (CD33, CD123 or another tumoral target, like but not restricted to CD19, Mesothelin, BCMA) and the IL-1 receptor type 2 (IL-1R2), as well as a method for modulating the tumoral microenvironment.

RfuCAR construct and mechanism is a novel approach that aims at regulating the tumoral microenvironment not only through the cell signaling pathway, but also to the secreted molecules from macrophages and other cells. In silico and in vitro tests are being conducted in order to corroborate the advantages of Celluris RfuCAR applications.

### BRIEF DESCRIPTION OF THE FIGURES

FIG. 1 presents the schematic of RfuCAR structure, wherein A is the vector scheme; and B is the Rfu scheme;

FIG. 2 presents the scheme demonstrating the switch and tuning of RfuCAR. The combination of peptides can switch-off or modulate the RfuCAR action. Peptides to IL-1R and Tumor Target, Peptides to IL-1R-IL-1 and Tumor Target and both three peptides can switch-off RfuCAR. The administration of only one of the peptides modulates the RfuCAR action;

FIG. 3 presents the schematic view of putative anti-CD33 RfuCAR;

FIG. 4 presents the predictive structures (RaptorX) for Anti-CD33 scFv domain, IL1-R2 extracellular domain and IgG4 Hinge-CH2CH3 domains;

FIG. 5 presents the predictive tridimensional models for the tested sequences of extracellular domain of anti-CD33 RfuCAR. The numbers in the boxes corresponds to the model number 1 to 6. Are shown the best matched model for each construction in the two softwares: IntFold and RaptorX;

FIG. 6 presents the quality plots for all the best matches for each model for CD33-RfuCAR models (1-6);

FIG. 7 presents the disorder plots for the prediction models of anti-CD33 RfuCAR. Each plot represents the disorder prediction for each model 1 to 6;

FIG. 8 presents the predicted binding sites for RfuCAR model 2;

FIG. 9 presents the schematic view of putative anti-CD123 RfuCAR;

FIG. 10 presents the predictive structures (RaptorX) for Anti-CD123 scFv domain, IL1-R2 extracellular domain and IgG4 Hinge-CH2CH3 domains;

FIG. 11 presents the predictive tridimensional models for the tested sequences of extracellular domain of anti-CD123 RfuCAR. The numbers in the boxes corresponds to the model number 7 to 12. Are shown the best matched model for each construction in the two softwares: IntFold and RaptorX;

FIG. 12 presents the quality plots for all the best matches for each model for CD123-RfuCAR models (7-12);

FIG. 13 presents the disorder plots for the prediction models of anti-CD123 RfuCAR. Each plot represents the disorder prediction for each model 7 to 12;

FIG. 14 presents the predicted binding sites for RfuCAR model 8.

### DETAILED DESCRIPTION OF THE INVENTION

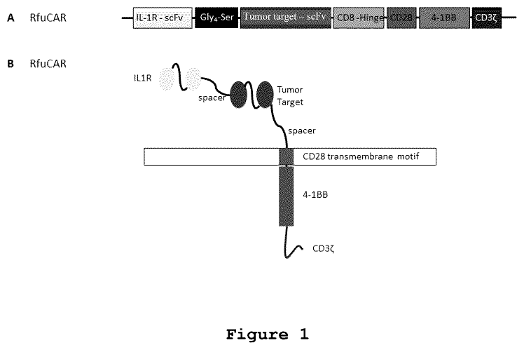

The present invention describes a bispecific in tandem receptor CAR, named RfuCAR, which includes a scFv that recognizes and ligates surface molecules on tumoral cells (CD33, CD123 or another tumoral target, like but not restricted to CD19, Mesothelin, BCMA) and the IL-1 receptor type 2 (IL-1R2), as well as a method for modulating the tumoral microenvironment. Additionally, such mechanism is able to be switched-off by the administration of both receptor epitope peptides, constituting a regulatory safety switch (FIGS. 1 A and B).

The scFv motif will ligate the tumoral cell and lead them to apoptosis exactly as a third generation CAR works. The secreted IL-1β will be trapped binding in the IL-1R of RfuCAR inhibiting the binding to the IL-1R1 and IL-1 signaling transduction. This inhibition will decrease the IL-1 pathway activation leading to the modulation of tumoral cell proliferation. Regulation of IL-1 content in the microenvironment will modulate other cytokines activation in the tumoral environment, putatively preventing the oversecretion of cytokines observed in CRS and neurotoxicity events.

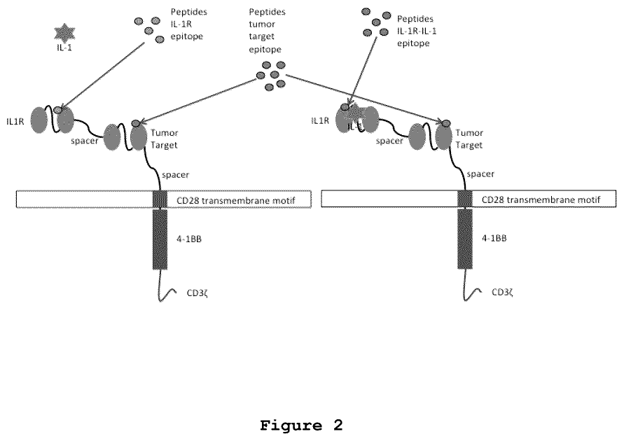

If any of the toxic CAR-T effects are detected in the patient, the RfuCAR cells action can be transiently switched-off and also tuned by the administration of different peptides that link to IL-1R, IL-1-IL-1R and/or scFv epitopes (FIG. 2). These peptides will be able to link the two RfuCAR preventing the ligation to tumoral cells and/or IL-1 inhibiting or modulating RfuCAR activity. The RfuCAR cells will still be able to proliferate in the patient and get back to kill tumoral cells as soon as the administration of the peptides ceases.

### RfuCAR Putative Structures

The extracellular putative structures of RfuCAR were tested in two structure prediction online features—IntFold (MCGUFFIN, et al. 2010; MCGUFFIN, et al. 2018; MCGUFFIN, et al. 2015; BUENAVISTA, et al. 2012; ROCHE, et al. 2012) and RaptorX (KALLBERG, et al. 2012; M A, et al. 2012; PENG, et al. 2011; PENG, et al. 2011; M A, et al. 2013). These features compare the submitted primary structure with characterized secondary and tertiary structures in the PDB database predicting the putative structure and given scores to indicate the similarity. Also, they provide disorder regions and putative binding sites.

For the data interpretation, the following points should be taken into account:
RaptorX
Score: is the alignment score falling between 0 and the (domain) sequence length, with 0 indicating the worst. In practice, Score may slightly go beyond the sequence length due to estimation error.
uSeqID and SeqID: is the number of identical residues in the alignment. SeqID is uSeqID normalized by the protein (or domain) sequence length and multiplied by 100. The higher the uSeqID (SeqID), the better. If the SeqID>30% and the protein (or domain) has >200 residues, it usually indicates that the predicted model has a correct fold.
uGDT and GDT: uGDT is the unnormalized GDT (Global Distance Test) score defined as 1*N(1)+0.75*N(2)+0.5*N(4)+0.25*N(8), where N(x) is the number of residues with estimated modeling error (in A) smaller than x. GDT is calculated as uGDT divided by the protein (or domain) length and multiplied by a 100. uGDT(GDT) measures the absolute model quality. For a protein with >100 residues, uGDT>50 is a good indicator. For a protein with <100 residues, GDT>50 is a good indicator. If a model has good uGDT (>50) but bad GDT (<50), it indicates that only a small portion of the model may be good.
P-value: is the likelihood of a predicted model being worse than the best of a set of randomly-generated models for this protein (or domain), so P-value evaluates the relative quality of a model. The smaller the P-value, the higher quality the model. For mainly alpha proteins, P-value less than 10-3 is a good indicator. For manly beta proteins, P-value less than 10-4 is a good indicator.
IntFold

The results table is ranked according to decreasing global model quality score. The global model quality scores range between 0 and 1. In general scores less than 0.2 indicate there may be incorrectly modelled domains and scores greater than 0.4 generally indicate more complete and confident models, which are highly similar to the native structure. Each model is also assigned a color coded confidence level depending on the p-value:

| P-value | | |
|---|---|---|
| cut-off | Confidence | Description |
| p < 0.001 | CERT | Less than a 1/1000 chance that the |
| | | model is incorrect. |
| p < 0.01 | HIGH | Less than a 1/100 chance that the model |
| | | is incorrect, |
| p < 0.05 | MEDIUM | Less than a 1/20 chance that the model |
| | | is incorrect. |
| p < 0.1 | LOW | Less than a 1/10 chance that the model |
| | | is incorrect. |
| P > 0.1 | POOR | Likely to be a poor model with little or |
| | | no similarity to the native structure. |

The confidence scores should be considered in conjunction with the local model quality (per-residue scores) and the coverage of the target protein by the template/templates. The per-residue scores indicate the predicted distance (in Angstroms) between the CA atom of the residue in the model and the CA atom of the equivalent residue in the native structure.
The 3D cartoon view of the model that is color-coded with the residue error according to the RasMol temperature coloring scheme.
Disorder prediction—The image shows a plot of the probability of disorder (on the y axis) for each numbered amino acid in the sequence (on the x axis). The disorder/order probability threshold is shown as a dashed line on the plot. Residues above the threshold could be considered as mostly disordered and below as mostly ordered, however this threshold serves only to guide the user.
Domain boundary prediction—The image shows the top predicted 3D model colored to indicate predicted domains
a change in color indicates a likely domain boundary.
Binding site prediction—The image shows the top predicted 3D model annotated to indicate putative binding site residues. The cartoon view of the model is shown in green and the binding site resides are shown as blue sticks with labeled residues. A list of the binding residues is provided along with the most likely (numerous) ligand, the ligand identified at nearest to the center of the predicted binding pocket and a list of the likely interacting ligands and the number of each that were identified in related template structures.

### RfuCAR Anti-CD33

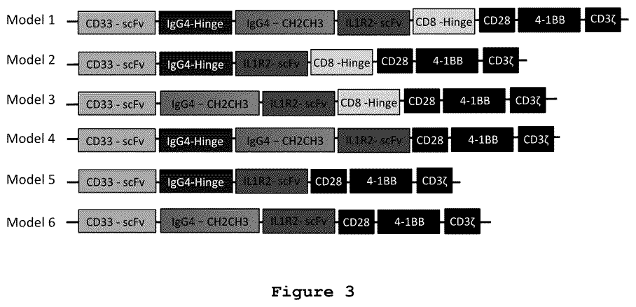

The tested models for RfuCAR anti-CD33 were described in the FIG. 3 and the sequences used are represented by SEQ. ID. Nos. 1 to 6. The schemes of colors are the same in the schematic view and in the sequences.

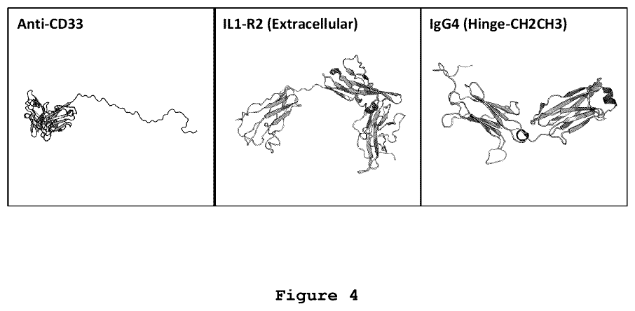

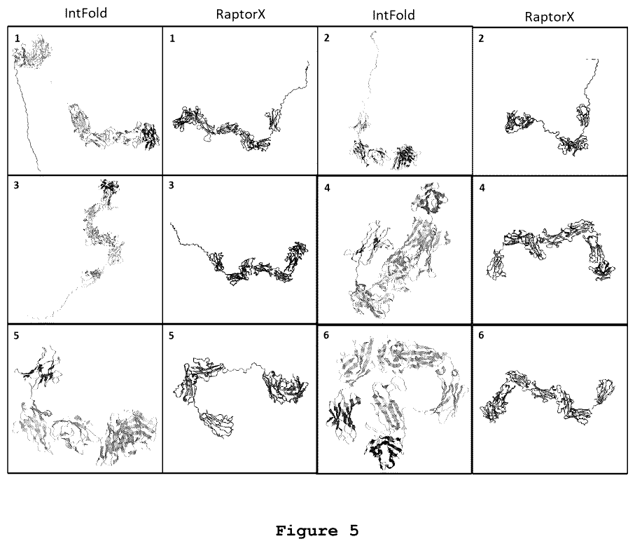

The tested sequences generated the following tridimensional predictive structures for each domain alone (FIG. 4) and for the putative constructions (FIG. 5).

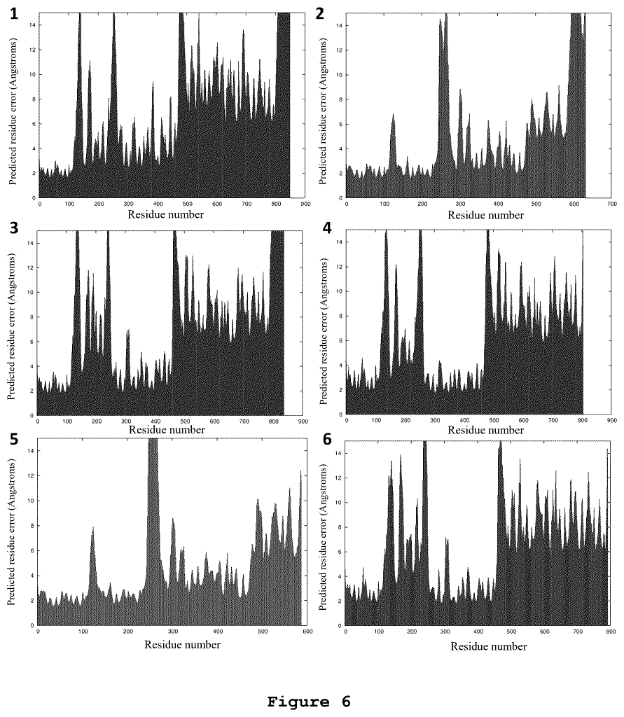

Apparently, the structure of model 2 (represented by SEQ. ID. No. 2) is the one that maintains the adequate structure of the included components. It is corroborated by the technical data reported by each software compiled in the tables 1 (Domains alone) and table 2 (Anti-CD33 RfuCAR models). The model 2 has the best quality score (0.4465) in IntFold, when compared to the other models. This score is not high but sufficient to indicate a good structural prediction. Also, the analysis of the quality plots (FIG. 6) for all models indicate that the mismatches between the primary sequence of the model and the matched templates are minor in the model 2, indicating a more accurate prediction.

TABLE 1
Results for predictive tridimensional structures for each component of RfuCAR tested
alone on RaptorX software.
RaptorX

| | | | | | | | Overall | Disordered |
|---|---|---|---|---|---|---|---|---|
| | Templates | | | | uSeqId | | uGDT | Positions |
| Component | Matched | Position | p-value | uGDT (GDT) | (SeqId): | Score | (GDT): | (number/%) |
| IL1-R2 Extracellular | 3o4o:C | 1-330 | 2.41e−13 | 263 (80) | 314 (95) | 218 | 263 (79) | 15 (4%) |
| IgG4 (Hinge-CH2CH3) | 5dk3:B | 1-229 | 2.53e−10 | 182 (80) | 212 (93) | 156 | 182 (79) | 24 (10%) |
| Anti-CD33 | 51xaH 6ehyA | 1-290 | 4.25e−11 | 194 (67) | 150 (52) | 69 | 193 (66) | 29 (10%) |
| | 5aawA | | | | | | | |
| Anti-CD123 | 2gki:A | 1-293 | 3.62e−12 | 193 (66) | 158 (54) | 171 | 192 (65) | 39 (13%) |

TABLE 2
Compiled results of predictive structure for models 1 to 6 anti-CD33 RfuCAR in the
softwares IntFold and RaptorX.

| | | | | RaptorX | | | | | | | | |
|---|---|---|---|---|---|---|---|---|---|---|---|---|
| | | | | | | | | | | | | Disordered |
| | IntFold | | | | | | | | | | Overall | Positions |
| | Templates | Confidence | Quality | | Templates | | | uGDT | uSeqId | | uGDT | (number/ |
| Model | Matched | p-value | Score | Domains | Matched | Position | p-value | (GDT) | (SeqId): | Score | (GDT): | %) |
| 1 | [symbol] , | MEDIUM | 0. [symbol] 8 | 2 | 5dk3:B | 1-476 | 3.13e−18 | 305 (64) | 311 (65) | 294 | 572 (67) | 32 (3%) |
| | 4yfcB | 2.01 [symbol] E−2 | | | 3o4o:C | 477-849 | 1.54e−13 | 68 (72) | 314 (84) | 220 | | |
| 2 | [symbol] , | 8.724E−4 | 0. [symbol] 65 | 2 | 3o4o:C | 267-632 | 1.95e−13 | 264 (72) | 314 (86) | 217 | 462 (73) | 21 (3%) |
| | 3o4oC | | | | [symbol] | 1-266 | 3.15e−11 | 199 (75) | 150 (66) | 169 | | |
| | | | | | 6 [symbol] | | | | | | | |
| | | | | | [symbol] | | | | | | | |
| 3 | lm [symbol] , | MEDIUM | 0. [symbol] 614 | 2 | 5dk3:B | 1-464 | 2.65e−18 | 300 (65) | 302 (65) | 298 | 566 (67) | 33 (3%) |
| | [symbol] | 1.4 [symbol] E−2 | | | 3o4o:C | 465-837 | 1.72e−13 | 266 (71) | 314 (84) | 221 | | |
| | [symbol] , | | | | | | | | | | | |
| | 3o4o:C | | | | | | | | | | | |
| 4 | [symbol] , | | 0.4055 | 2 | 5dk3:B | 1-476 | 5.01e−18 | 306 (64) | 314 (66) | 293 | 567 (70) | 32 (3%) |
| | 3o4o:C | 5.143E−3 | | | 3o4o:C | 477-804 | 2.31e−13 | 262 (80) | 314 (98) | 220 | | |
| 5 | [symbol] , | 1.095E−3 | 0.4412 | 2 | 3o4o:C | | 3.27e−13 | 261 (81) | 314 (98) | 220 | 466 (77) | 22 (3%) |
| | 3o4oC | | | | [symbol] H | 267-687 | 3.53e−11 | 196 (74) | 148 (66) | 167 | | |
| | | | | | 6 [symbol] | 1-266 | | | | | | |
| | | | | | [symbol] A | | | | | | | |
| 6 | [symbol] , | 5.677E−3 | 0.4032 | 2 | 5dk3:B | 1-464 | 8.19e−18 | 308 (66) | 302 (65) | 298 | 569 (71) | 32 (4%) |
| | 4yfcB | | | | 3o4o:C | 465-792 | 2.58e−13 | 261 (80) | 314 (96) | 319 | | |

[symbol]

indicates data missing or illegible when filed

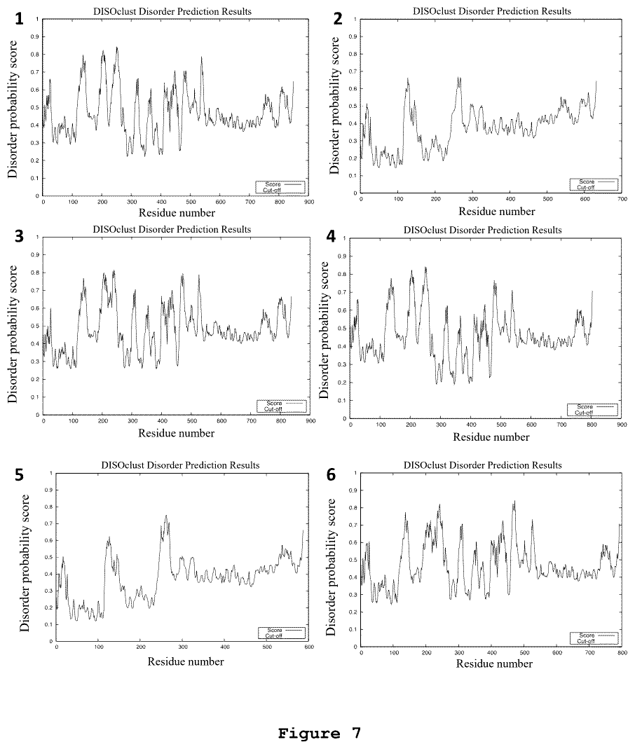

The model 2 has the best results of predicted structure in both softwares. The templates matched in the database are also more similar to the function expected for RfuCAR. Model 2 has matched to the structure of IL-1 receptor complex and also to an scFv motif (Table 3 reports all of the matched templates), indicating that probably the structure of the anti-CD33 and the IL-1R2 receptor are maintained in this model. The spacer applied in this model is only the Hinge of IgG4 but considering the percentage and regions of disorder (table 2, FIG. 7) it seems to allow the adequate mobility of the motifs. The most disordered regions, those regions without a regular secondary structure and more flexible, are in the IgG4 Hinge region and in the linker between the two chains on the scFv (anti-CD33) region. The other models present more disorder regions, that can indicate a less accurate predictive model or a protein with a tertiary structure that is not similar to the expected.

TABLE 3
Matched templates for all the predicted models of RfuCAR

| Template | Description |
|---|---|
| 3o4oC | Interleukin-1 receptor complex-C chain |
| 5dk3:B | IgG4 Antibody-B chain |
| 51xaH | Adiponectin receptor 2 |
| 6ehyA | scFv AbVance (Generic scFv)-A chain |
| 6ehxB | B-chain |
| 5aawA | Structure of a redesigned cross-reactive antibody to |
| | dengue virus-A chain |
| 2qki:A | Heavy and light chain variable single domains of an |
| 2gkiB | anti-DNA binding antibody-A chain |
| | Bchain |
| 1hzhH | Human igg b12-Heavy chain |
| 4yfcB | Interleukin-1 receptor accessory |
| | protein-like 1. Chain: b. |
| 5yd5A | scFv antibody 4b08 with epitope peptid-A chain |
| 2ghwB | Sars spike protein receptor binding domain in |
| | complex with a neutralizing antibody-B chain |
| 5b6fB | Fab fragment of an anti-leukotriene c4 monoclonal |
| | antibody-B chain |
| 1l6xA | Fc fragment of rituximab (anti-CD20)-A-chain |
| 3ay4A | Nonfucosylated fc complexed with bis-glycosylated |
| | soluble form of fc gamma receptor iiia-A chain |
| 4pp1D | der p 1 allergen complexed with fab fragment of |
| | mab 5h8-D chain |
| 1igtD | Igg2a intact antibody-mab231. D chain |
| 1igtB | B chain |
| 1mcoH | Human immunoglobulin with a hinge deletion-Heavy |
| | Chain |
| 1r70B | Human iga2 (m1) light chain-B-chain |

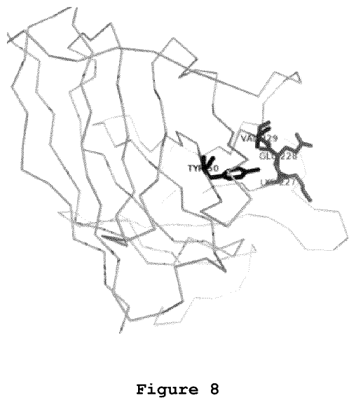

The predicted binding sites and locations for the Model 2 are positions: 50, 227, 228, 229; 272 and 360. Most likely ligands at each site (Type): ILE; FUL. Centroid ligands at each site (TypeID): SER657; FUL641. All ligands in clusters (Type-Frequency): GLY-1, TRP-1, GLU-2, ILE-3, PRO-3, SER-2, THR-2, TYR-3, ASP-1, LEU-1, ASN-1; FUL-1, FUC-1. Likely+centroid ligands at each site: ILE650; FUL641. The predicted binding sites are shown in the FIG. 8.

### RfuCAR Anti-CD123

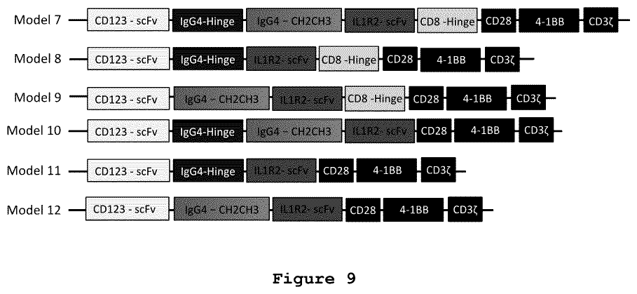

The tested models for RfuCAR anti-CD123 were described in the FIG. 9 and the sequences used are represented by SEQ. ID. Nos. 7 to 12. The schemes of colors are the same in the schematic view and in the sequences.

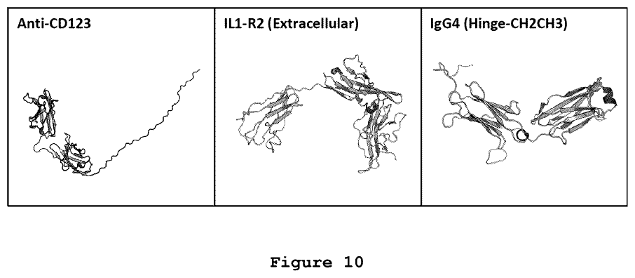

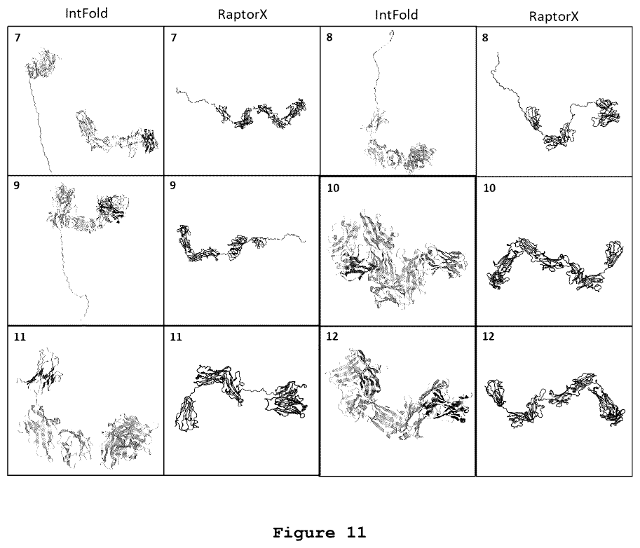

The tested sequences generated the following tridimensional predictive structures for each domain alone (FIG. 10) and for the putative constructions (FIG. 11).

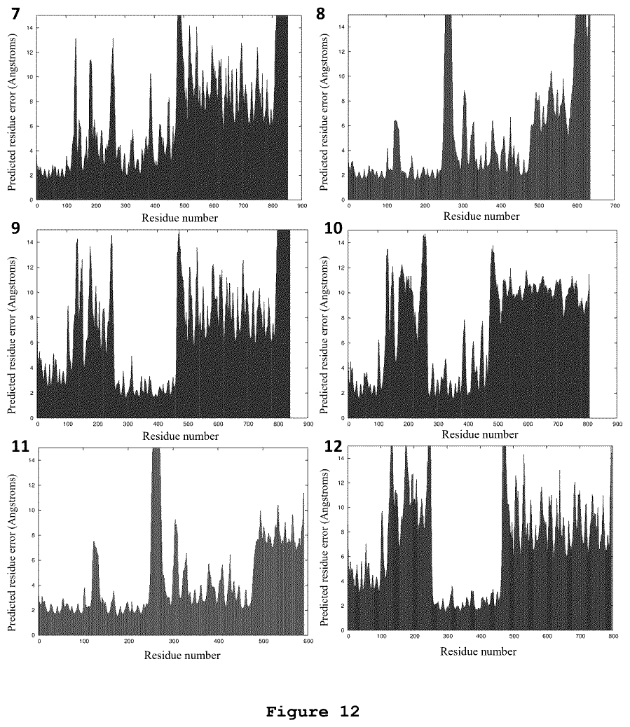

Apparently, the structure of model 8 (represented by SEQ. ID. No. 8) is the one that maintains the adequate structure of the included components. It is corroborated by the technical data reported by each software compiled in the tables 1 (Domains alone) and 4 (Anti-CD123 RfuCAR models). This model has the second best quality score on IntFold analysis (0.4404). The model 8 quality score is minor than the model 11 and the quality plots have very similar distribution (FIG. 12). Despite this, model 8 maintains the CD8 hinge near the cells membrane as constructed for the anti-CD33 and anti-CD123 CAR-Ts, that have been already proven to be functional. The model 11 does not have the CD8 hinge region.

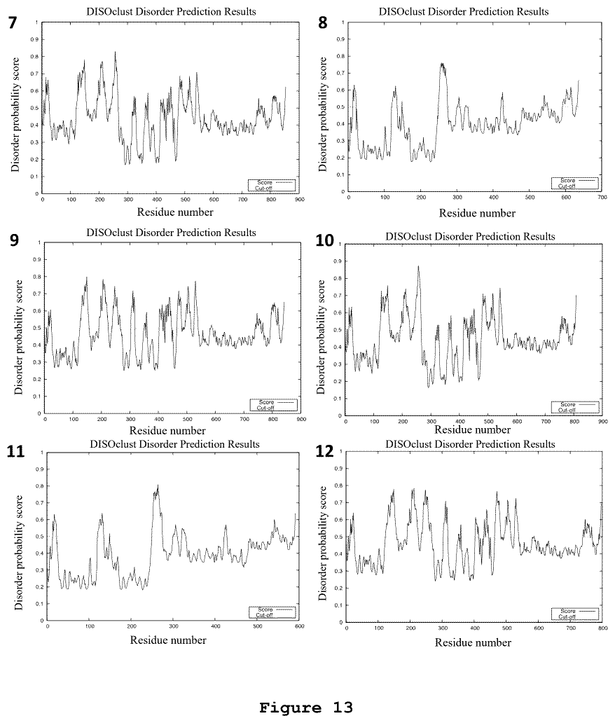

The model 8 has also one of the best results in RaptorX software. The templates matched in the database are more similar to the function expected for RfuCAR. Model 8 has matched to the structure of IL-1 receptor complex and also to an scFv motif (Table 3 reports all of the matched templates), indicating that probably the structure of the anti-CD123 and the IL-1R2 receptor are maintained in this model. The spacer applied in this model is only the Hinge of IgG4 but considering the percentage and regions of disorder (table 11, FIG. 13) it seems to allow the adequate mobility of the motifs. The most disordered regions, those regions without a regular secondary structure and more flexible, are in the IgG4 Hinge region and in the linker between the two chains on the scFv (anti-CD123) region. The models 7, 9, 10 and 12 have many highly disordered regions, indicating regions without good predictive fold.

TABLE 11
Compiled results of predictive structure for models 8 to 12 anti-CD123 RfuCAR in
in the softwares IntFold and RaptorX.

| | | | | RaptorX | | | | | | | | |
|---|---|---|---|---|---|---|---|---|---|---|---|---|
| | | | | | | | | | | | | Disordered |
| | IntFold | | | | | | | | | | Overall | Positions |
| | Templates | Confidence | Quality | Do- | Templates | | | uGDT | uSeqId | | uGDT | (number/ |
| Model | Matched | p-value | Score | mains | Matched | Position | p-valve | (GDT) | (SeqId): | Score | (GDT): | [symbol] ) |
| 7 | [symbol] , | MEDIUM | 0.37 [symbol] 9 | 2 | 5dk3 [symbol] | 1-480 | 1.0 [symbol] e−18 | [symbol] 17 (66) | 30 [symbol] ) | [symbol] | 581 (6 [symbol] ) | [symbol] (28) |
| | [symbol] | 1.5 [symbol] −2 | | | 3o4o:C | 451-853 | 1.74e−13 | 260 ( [symbol] 1) | [symbol] 14 (34) | 2 [symbol] 0 | | |
| 8 | [symbol] , | 1.137E−3 | 0. [symbol] 04 | 2 | 3o4o:C | 271-636 | 1.05e−13 | 266 (73) | [symbol] 14 (66) | 223 | [symbol] 48 (70) | 24(3 [symbol] ) |
| | [symbol] , | | | | 6ehx [symbol] | 1- | [symbol] .05e−12 | 183 (68) | 153( [symbol] 7) | [symbol] 70 | | |
| | 3o4oC, | | | | 5 [symbol] A | 27 [symbol] | | | | | | |
| | [symbol] | | | | | | | | | | | |
| 9 | 5 [symbol] B, | MEDIUM | 0. [symbol] 762 | 2 | [symbol] | 1-46 [symbol] | 1.0 [symbol] e−18 | 306( [symbol] ) | 205(63) | 307 | 57 [symbol] ) | 24(2 [symbol] ) |
| | 116xA, | 1. [symbol] 08E−2 | | | 3o4o:C | 469-841 | 1. [symbol] e−1 [symbol] | 2 [symbol] 6(71) | 314 ( [symbol] 4) | 2 [symbol] 1 | | |
| | 4 [symbol] | | | | | | | | | | | |
| 10 | [symbol] , | 1. [symbol] 3 | 0.3 [symbol] 49 | 2 | 5dk3:B | 1-480 | 5.46e−19 | 318(66) | 307 (64) | 203 | 582 (7 [symbol] ) | 2 [symbol] ) |
| | [symbol] , | | | | 3o4o:C | 481- | 1.27e−15 | 365( [symbol] ) | [symbol] 14 (96) | 221 | | |
| | [symbol] | | | | | [symbol] 08 | | | | | | |
| 11 | 5yd5A, | 3. [symbol] 6E−4 | 0.4656 | 2 | 3o4o:C | 371-591 | 1. [symbol] e−13 | 263( [symbol] ) | 314 (98) | 221 | 450 (76) | 2 [symbol] ) |
| | 3o4oC, | | | | 6 [symbol] | 1-270 | 8 [symbol] 77e−12 | 1 [symbol] 7 (69) | 153( [symbol] 7) | 168 | | |
| 12 | [symbol] | 9.326E−3 | 0.3900 | 2 | [symbol] | 1-468 | 1.70e−18 | 309(66) | 2 [symbol] 5(63) | 30 [symbol] | 571 (71) | 24(3 [symbol] ) |
| | [symbol] | | | | 3o4o:C | 469-796 | 2.98e−13 | 263( [symbol] ) | 314 (96) | 315 | | |
| | 4 [symbol] | | | | | | | | | | | |

[symbol]

indicates data missing or illegible when filed

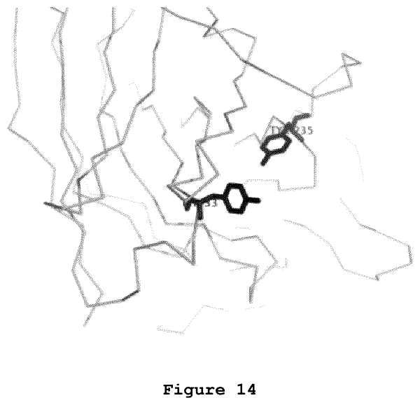

The predicted binding sites and locations for the Model 8 are positions: 33, 235. Most likely ligands at each site (Type): TYR. Centroid ligands at each site (TypeID): PRO656. All ligands in clusters (Type-Frequency): GLY-3, TRP-2, GLU-2, ILE-3, PRO-4, SER-3, ARG-1, THR-2, TYR-5, ASP-1, LEU-1, ASN-4, ALA-2, CYS-1. Likely+centroid ligands at each site: TYR672. The predicted binding sites are shown in the FIG. 14.

Therefore, according to the reported predictive structures the best choice of spacer, those which have the better chance to maintain in vivo the structure of the domains in RfuCAR are the model 2 (SEQ. ID. No. 2) for anti-CD33 RfuCAR and model 8 (SEQ. ID. No. 8) for anti-CD123 RfuCAR. Both include the anti-tumoral scFv, the IL1-R2 receptor with IgG4 Hinge as a spacer between receptors and CD8 hinge as a spacer from cell membrane.

Although the invention has been amply described, it is obvious to those skilled in the art that various changes and modifications may be made to improve the design without such changes being outside the scope of the invention.

### BIBLIOGRAPHIC REFERENCES

DINARELLO CA. Biologic Basis For Interleukin-1 In Disease. Blood. 1996; 87(6):2095-2147.
APTE RN, Voronov E. Interleukin-1-A Major Pleiotropic Cytokine In Tumor-Host Interactions. Semin Cancer Biol. 2002; 12(4):277-290. Doi:10.1016/S1044-579X(02)00014-7.
DUNN E, Sims J E, Nicklin M J H, O'Neill L A J. Annotating Genes With Potential Roles In The Immune System: Six New Members Of The IL-1 Family. Trends Immunol. 2001; 22(10):533-536. Doi:10.1016/S1471-4906(01)02034-8.
DINARELLO C, Arend W, Sims J, Smith D, Blumberg H, O'Neill L. IL-1 Family Nomenclature. Nat Immunol. 2010; 11(11):973. Doi:10.1038/Oby.2010.55.
WEBER A, Wasiliew P, Kracht M. Interleukin-1 (IL-1) Pathway. Sci Signal. 2010; 3(105). Doi:10.1126/Scisignal.3105cm1.
AURON PE. The Interleukin 1 Receptor: Ligand Interactions And Signal Transduction. Cytokine Growth Factor Rev. 1998; 9(3-4):221-237. Doi:10.1016/S1359-6101(98)00018-5.
VIDAL-VANACLOCHA F, Alvarez A, Asumendi A, Urcelay B, Tonino P, Dinarello C A. Interleukin 1 (IL-1)-Dependent Melanoma Hepatic Metastasis In Vivo; Increased Endothelial Adherence By IL-1-Induced Mannose Receptors And Growth Factor Production In Vitro. J Natl Cancer Inst. 1996; 88 (3-4):198-205. Doi:10.1093/Jnci/88.3-4.198.
COXON A, Bolon B, Estrada J, Et Al. Inhibition Of Interleukin-1 But Not Tumor Necrosis Factor Suppresses Neovascularization In Rat Models Of Corneal Angiogenesis And Adjuvant Arthritis. Arthritis Rheum. 2002; 46(10):2604-2612. Doi:10.1002/Art.10546.
VORONOV E, Shouval D S, Krelin Y, Et Al. IL-1 Is Required For Tumor Invasiveness And Angiogenesis. Proc Natl Acad Sci. 2003; 100(5):2645-2650. Doi:10.1073/Pnas.0437939100.
SONG X, Voronov E, Dvorkin T, Et Al. Differential Effects Of IL-1 And IL-1 On Tumorigenicity Patterns And Invasiveness. J Immunol. 2003; 171(12):6448-6456. Doi:10.4049/Jimmunol.171.12.6448.
CAREY A, Edwards D K, Eide C A, Et Al. Identification Of Interleukin-1 By Functional Screening As A Key Mediator Of Cellular Expansion And Disease Progression In Acute Myeloid Leukemia. Cell Rep. 2017; 18(13):3204-3218. Doi:10.1016/J.Celrep.2017.03.018.
BONECCHI R, Garlanda C, Mantovani A, Riva F. Cytokine Decoy And Scavenger Receptors As Key Regulators Of Immunity And Inflammation. Cytokine. 2016; 87:37-45. Doi:10.1016/J.Cyto.2016.06.023.
NORELLI M, Camisa B, Barbiera G, Et Al. Monocyte-Derived I L-1 And IL-6 Are Differentially Required For Cytokine-Release Syndrome And Neurotoxicity Due To CAR T Cells. Nat Med. May 2018:1. Doi:10.1038/541591-018-0036-4.
GIAVRIDIS T, Van Der Stegen S J C, Eyquem J, Hamieh M, Piersigilli A, Sadelain M. CAR T Cell-Induced Cytokine Release Syndrome Is Mediated By Macrophages And Abated By IL-1 Blockade. Nat Med. 2018:1. Doi:10.1038/541591-018-0041-7.
TARASEVICIUTE A, Tkachev V, Ponce R, Et Al. Chimeric Antigen Receptor T Cell-Mediated Neurotoxicity In Nonhuman Primates. Cancer Discov. March 2018:CD-17-1368. Doi:10.1158/2159-8290.CD-17-1368.
LUST JA, Lacy M Q, Zeldenrust S R, Et Al. Induction Of A Chronic Disease State In Patients With Smoldering Or Indolent Multiple Myeloma By Targeting Interleukin 1B-Induced Interleukin 6 Production And The Myeloma Proliferative Component. Mayo Clin Proc. 2009; 84(2):114-122. Doi:10.4065/84.2.114.
HALLEGUA DS. Potential Therapeutic Uses Of Interleukin 1 Receptor Antagonists In Human Diseases. Ann Rheum Dis. 2002; 61(11):960-967. Doi:10.1136/Ard.61.11.960.
DINARELLO CA. Why Not Treat Human Cancer With Interleukin-1 Blockade? Cancer Metastasis Rev. 2010; 29(2):317-329. Doi:10.1007/510555-010-9229-0.
DINARELLO C A, Thompson R C. Blocking IL-1: Interleukin 1 Receptor Antagonist In Vivo And In Vitro. Immunol Today. 1991; 12(11):404-410. Doi:10.1016/0167-5699(91)90142-G.
MANTOVANI A, MUZIO M, GHEZZI P, COLOTTA C, INTRONA M. Regulation Of Inhibitory Pathways Of The Interleukin-1 System. Ann N Y Acad Sci. 1998; 840(1):338-351. Doi:10.1111/J.1749-6632.1998.Tb09573.X.
COLOTTA F, Re F, Muzio M, Et Al. Interleukin-1 Type II Receptor: A Decoy Target For IL-1 That Is Regulated By IL-4. Science (80-). 1993; 261(5120):472-475. Doi:10.1126/Science.8332913.
MCMAHAN CJ, Slack J L, Mosley B, Et Al. A Novel IL-1 Receptor, Cloned From B Cells By Mammalian Expression, Is Expressed In Many Cell Types. EMBO J. 1991; 10(10):2821-2832. Http://Www.Pubmedcentral.Nih.Gov/Articlerender.Fcgi?Artid=452 992&Tool=Pmcentrez&Rendertype=Abstract.
THOMAS C, Bazan J F, Garcia K C. Structure Of The Activating IL-1 Receptor Signaling Complex. 2014; 19(4):455-457. Doi:10.1038/Nsmb.2260.Structure.
SYMONS JA, Young P R, Duff G W. Soluble Type-Ii Interleukin-1 (Il-1) Receptor Binds And Blocks Processing Of Il-1-Beta Precursor And Loses Affinity For Il-1 Receptor Antagonist. Proc Natl Acad Sci USA. 1995; 92(5):1714-1718. Doi:DOI 10.1073/Pnas.92.5.1714.
BOURKE E, Cassetti A, Villa A, Fadlon E, Colotta F, Mantovani A. IL-1 Scavenging By The Type II IL-1 Decoy Receptor In Human Neutrophils. J Immunol. 2003; 170 (12):5999-6005. Doi:10.4049/Jimmunol.170.12.5999.
RE F, Muzio M, De Rossi M, Et Al. The Type II “Receptor” As A Decoy Target For Interleukin 1 In Polymorphonuclear Leukocytes: Characterization Of Induction By Dexamethasone And Ligand Binding Properties Of The Released Decoy Receptor. J Exp Med. 1994; 179(2):739-743. Doi:10.1084/Jem.179.2.739.
RAUSCHMAYR T, Groves R W, Kupper T S. Keratinocyte Expression Of The Type 2 Interleukin 1 Receptor Mediates Local And Specific Inhibition Of Interleukin 1-Mediated Inflammation. Med Sci. 1997; 94 (May):5814-5819.
BESSIS N, Guery L, Mantovani A, Et Al. The Type II Decoy Receptor Of IL-1 Inhibits Murine Collagen-Induced Arthritis. Eur J Immunol. 2000; 30 (3):867-875. Doi:10.1002/1521-4141(200003)30:3<867::AID-IMMU867>3.0.CO;2-M.
DAWSON J, Engelhardt P, Kastelic T, Cheneval D, Mackenzie A, Ramage P. Effects Of Soluble Interleukin-1 Type II Receptor On Rabbit Antigen-Induced Arthritis: Clinical, Biochemical And Histological Assessment. Rheumatology (Oxford). 1999; 38(5):401-406. Http://Www.Ncbi.Nlm.Nih.Gov/Pubmed/10371276.
ATTUR MG, Dave M, Cipolletta C, Et Al. Reversal Of Autocrine And Paracrine Effects Of Interleukin 1 (IL-1) In Human Arthritis By Type II IL-1 Decoy Receptor. Potential For Pharmacological Intervention. J Biol Chem. 2000; 275(51):40307-40315. Doi:10.1074/Jbc.M002721200.
KHOUFACHE K, Bondza P K, Harir N, Et Al. Soluble Human IL-1 Receptor Type 2 Inhibits Ectopic Endometrial Tissue Implantation And Growth: Identification Of A Novel Potential Target For Endometriosis Treatment. Am J Pathol. 2012; 181(4):1197-1205. Doi:10.1016/J.Ajpath.2012.06.022.
BELLEHUMEUR C, Collette T, Maheux R, Mailloux J, Villeneuve M, Akoum A. Increased Soluble Interleukin-1 Receptor Type II Proteolysis In The Endometrium Of Women With Endometriosis. Hum Reprod. 2005; 20(5):1177-1184. Doi:10.1093/Humrep/Deh749.
GUAY S, Akoum A. Stable Inhibition Of Interleukin 1 Receptor Type II In Ishikawa Cells Augments Secretion Of Matrix Metalloproteinases: Possible Role In Endometriosis Pathophysiology. Reproduction. 2007; 134(3):525-534. Doi:10.1530/REP-06-0377.
SIMEONI E, Dudler J, Fleury S, Et Al. Gene Transfer Of A Soluble IL-1 Type 2 Receptor-Ig Fusion Protein Improves Cardiac Allograft Survival In Rats. Eur J Cardiothorac Surg. 2007; 31(2):222-228. Doi:10.1016/J.Ejcts.2006.10.042.
CHANG H, Wang Y, Wu W, Li G, Hanawa H, Zou J. Hydrodynamics-Based Delivery Of An Interleukin-1 Receptor II Fusion Gene Ameliorates Rat Autoimmune Myocarditis By Inhibiting IL-1 And Th17 Cell Polarization. Int J Mol Med. 2013; 31(4):833-840. Doi:10.3892/Ijmm.2013.1276.
BOSSÚ P, Visconti U, Ruggiero P, Et Al. Transfected Type II Interleukin-1 Receptor Impairs Responsiveness Of Human Keratinocytes To Interleukin-1. Am J Pathol. 1995; 147(6):1852-1861. Http://Www.Pubmedcentral.Nih.Gov/Articlerender.Fcgi?Artid=1 869949&Tool=Pmcentrez&Rendertype=Abstract.
MAR AC, Chu C H, Lee H J, Et Al. Interleukin-1 Receptor Type 2 Acts With C-Fos To Enhance The Expression Of Interleukin-6 And Vascular Endothelial Growth Factor A In Colon Cancer Cells And Induce Angiogenesis. J Biol Chem. 2015; 290(36):22212-22224. Doi:10.1074/Jbc.M115.644823.
CHMIELEWSKI M, Hombach A A, Abken H. Antigen-Specific T-Cell Activation Independently Of The MHC: Chimeric Antigen Receptor-Redirected T Cells. Front Immunol. 2013; 4(November):1-7. Doi:10.3389/Fimmu.2013.00371.
HUDECEK M, Sommermeyer D, Kosasih P L, Et Al. The Nonsignaling Extracellular Spacer Domain Of Chimeric Antigen Receptors Is Decisive For In Vivo Antitumor Activity. Cancer Immunol Res. 2015; 3(2):125-135. Doi:10.1158/2326-6066.CIR-14-0127.
GRADA Z, Hegde M, Byrd T, Et Al. Tancar: A Novel Bispecific Chimeric Antigen Receptor For Cancer Immunotherapy. Mol Ther—Nucleic Acids. 2013; 2 (April):E105. Doi:10.1038/Mtna.2013.32.
HEGDE M, Mukherjee M, Grada Z, Et Al. Tandem CAR T Cells Targeting HER2 And IL13RA 2 Mitigate Tumor Antigen Escape. J Clin Invest. 2016; 126(8):3036-3052. Doi:10.1172/JCI83416.We.
WATANABE N, Bajgain P, Sukumaran S, Et Al. Fine-Tuning The CAR Spacer Improves T-Cell Potency. Oncoimmunology. 2016; 5(12):E1253656. Doi:10.1080/2162402X.2016.1253656.
NORELLI M, Casucci M, Bonini C, Bondanza A. Clinical Pharmacology Of CAR-T Cells: Linking Cellular Pharmacodynamics To Pharmacokinetics And Antitumor Effects. Biochim Biophys Acta—Rev Cancer. 2016; 1865(1):90-100. Doi:10.1016/J.Bbcan.2015.12.001.
HOMBACH A, Heuser C, Gerken M, Et Al. T Cell Activation By Recombinant Fcεri Γ-Chain Immune Receptors: An Extracellular Spacer Domain Impairs Antigen-Dependent T Cell Activation But Not Antigen Recognition. Gene Ther. 2000; 7(12):1067-1075. Doi:10.1038/Sj.Gt.3301195.
GUEST RD, Hawkins R E, Kirillova N, Et Al. The Role Of Extracellular Spacer Regions In The Optimal Design Of Chimeric Immune Receptors. J Immunother. 2005; 28(3):203-211. Doi:10.1097/01.Cji.0000161397.96582.59.
JAMES SE, Greenberg P D, Jensen M C, Et Al. Antigen Sensitivity Of CD22-Specific Chimeric TCR Is Modulated By Target Epitope Distance From The Cell Membrane. J Immunol. 2008; 180 (10):7028-7038. Doi:10.4049/Jimmunol.180.10.7028.
DAVIS SJ, Van Der Merwe P A. The Kinetic-Segregation Model: TCR Triggering And Beyond. Nat Immunol. 2006; 7(8):803-809. Doi:10.1038/Ni1369.
PORTER DL, Levine B L, Kalos M, Bagg A, June C H. Chimeric Antigen Receptor-Modified T Cells In Chronic Lymphoid Leukemia. N Engl J Med. 2011; 365(8):725-733. Doi:10.1056/Nejmoa1103849.
KALOS M, Levine B L, Porter D L, Et Al. T Cells With Chimeric Antigen Receptors Have Potent Antitumor Effects And Can Establish Memory In Patients With Advanced Leukemia. Sci Transl Med. 2011; 3(95):95ra73-95ra73. Doi:10.1126/Scitranslmed.3002842.
SAVOLDO B, Ramos C A, Liu E, Et Al. CD28 Costimulation Improves Expansion And Persistence Of Chimeric Antigen Receptor-Modified T Cells In Lymphoma Patients. J Clin Invest. 2011; 121(5):1822-1826. Doi:10.1172/JCI46110.
REN-HEIDENREICH L, Hayman G T, Trevor K T. Specific Targeting Of EGP-2+Tumor Cells By Primary Lymphocytes Modified With Chimeric T Cell Receptors. Hum Gene Ther. 2000; 11(1):9-19. Doi:10.1089/10430340050016111.
MORITZ D, Groner B. A Spacer Region Between The Single Chain Antibody—And The CD3 Zeta-Chain Domain Of Chimeric T Cell Receptor Components Is Required For Efficient Ligand Binding And Signaling Activity. Gene Ther. 1995; 2 (8):539-546. Http://Www.Ncbi.Nlm.Nih.Gov/Pubmed/8593604.
PATEL SD, Moskalenko M, Smith D, Maske B, Finer M H, Mcarthur J G. Impact Of Chimeric Immune Receptor Extracellular Protein Domains On T Cell Function. Gene Ther. 1999; 6(3):412-419. Doi:10.1038/Sj.Gt.3300831.
HAWKINS RE, Ed. Cellular Therapy Of Cancer: Development Of Gene Therapy Based Approaches. World Scientific; 2014.
MATSUSHIMA N, Yoshida H, Kumaki Y, Et Al. Flexible Structures And Ligand Interactions Of Tandem Repeats Consisting Of Proline, Glycine, Asparagine, Serine, And/Or Threonine Rich Oligopeptides In Proteins. Curr Protein Pept Sci. 2008; 9(6):591-610. Doi:10.2174/138920308786733886.
VIDARSSON G, Dekkers G, Rispens T. Igg Subclasses And Allotypes: From Structure To Effector Functions. Front Immunol. 2014; 5(October):1-17. Doi:10.3389/Fimmu.2014.00520.
ALMASBAK H, Walseng E, Kristian A, Et Al. Inclusion Of An Igg1-Fc Spacer Abrogates Efficacy Of CD19 CAR T Cells In A Xenograft Mouse Model. Gene Ther. 2015; 22(5):391-403. Doi:10.1038/Gt.2015.4.
ANURATHAPAN U, Chan R C, Hindi H F, Et Al. Kinetics Of Tumor Destruction By Chimeric Antigen Receptor-Modified T Cells. Mol Ther. 2014; 22(3):623-633. Doi:10.1038/Mt.2013.262.
CASUCCI M, Falcone L, Camisa B, Bonini C, Bondanza A. CAR Spacers Including NGFR Domains Allow Efficient T-Cell Tracking And Mediate Superior Antitumor Effects. Mol Ther. 2015; 23 (May):S163-S164. Doi:10.1016/51525-0016(16)34023-0.
CASUCCI M, Falcone L, Camisa B, Et Al. Extracellular NGFR Spacers Allow Efficient Tracking And Enrichment Of Fully Functional CAR-T Cells Co-Expressing A Suicide Gene. Front Immunol. 2018; 9(March). Doi:10.3389/Fimmu.2018.00507.
WANG G, De Jong RN, Van Den Bremer E T J, Et Al. Molecular Basis Of Assembly And Activation Of Complement Component C1 In Complex With Immunoglobulin G1 And Antigen. Mol Cell. 2016; 63(1):135-145. Doi:10.1016/J.Molcel.2016.05.016.
AALBERSE RC, Schuurman J. Igg4 Breaking The Rules. Immunology. 2002; 105(1):9-19. Doi:10.1046/J.0019-2805.2001.01341.X.
QIN L, Lai Y, Zhao R, Et Al. Incorporation Of A Hinge Domain Improves The Expansion Of Chimeric Antigen Receptor T Cells. J Hematol Oncol. 2017; 10(1):68. Doi:10.1186/S13045-017-0437-8.
JONNALAGADDA M, Mardiros A, Urak R, Et Al. Chimeric Antigen Receptors With Mutated Igg4 Fc Spacer Avoid Fc Receptor Binding And Improve T Cell Persistence And Antitumor Efficacy. Mol Ther. 2015; 23(4):757-768. Doi:10.1038/Mt.2014.208.
MONTANO RF, Morrison S L. Influence Of The Isotype Of The Light Chain On The Properties Of Igg. J Immunol. 2002; 168 (1):224-231. Doi:10.4049/Jimmunol.168.1.224.
DORAI H, Wesolowski J S, Gillies S D. Role Of Inter-Heavy And Light Chain Disulfide Bonds In The Effector Functions Of Human Immunoglobulin Igg1. Mol Immunol. 1992; 29(12):1487-1491. Doi:10.1016/0161-5890(92)90222-J.
ALABANZA L, Pegues M, Geldres C, Et Al. Function Of Novel Anti-CD19 Chimeric Antigen Receptors With Human Variable Regions Is Affected By Hinge And Transmembrane Domains. Mol Ther. 2017; 25 (11):2452-2465. Doi:10.1016/J.Ymthe.2017.07.013.
JENA B, Dotti G, Cooper L J N. Redirecting T-Cell Specificity By Introducing A Tumor-Specific Chimeric Antigen Receptor. Blood. 2010; 116(7):1035-1044. Doi:10.1182/Blood-2010-01-043737.
KENDERIAN SS, Ruella M, Shestova O, Et Al. CD33-Specific Chimeric Antigen Receptor T Cells Exhibit Potent Preclinical Activity Against Human Acute Myeloid Leukemia. Leukemia. 2015; 29(8):1637-1647. Doi:10.1038/Leu.2015.52.
MARDIROS A, Forman S J, Budde L E. T Cells Expressing CD123 Chimeric Antigen Receptors For Treatment Of Acute Myeloid Leukemia. Curr Opin Hematol. 2015. Doi:10.1097/MOH.0000000000000190.
THOKALA R, Olivares S, Mi T, Et Al. Redirecting Specificity Of T Cells Using The Sleeping Beauty System To Express Chimeric Antigen Receptors By Mix-And-Matching Of VL And VH Domains Targeting CD123+Tumors. Unutmaz D, Ed. Plos One. 2016; 11(8):E0159477. Doi:10.1371/Journal.Pone.0159477.
LABORDA E, Mazagova M, Shao S, Et Al. Development Of A Chimeric Antigen Receptor Targeting C-Type Lectin-Like Molecule-1 For Human Acute Myeloid Leukemia. Int J Mol Sci. 2017; 18(11):2259. Doi:10.3390/Ijms18112259.
KENDERIAN SS, Porter D L, Gill S. Chimeric Antigen Receptor T Cells And Hematopoietic Cell Transplantation: How Not To Put The CART Before The Horse. Biol Blood Marrow Transplant. 2017; 23(2):235-246. Doi:10.1016/J.Bbmt.2016.09.002.
MCGUFFIN LJ, Roche D B. Rapid Model Quality Assessment For Protein Structure Predictions Using The Comparison Of Multiple Models Without Structural Alignments. Bioinformatics. 2010; 26(2):182-188. Doi:10.1093/Bioinformatics/Btp629.
MCGUFFIN LJ, Shuid A N, Kempster R, Et Al. Accurate Template-Based Modeling In CASP12 Using The Intfold4-TS, Modfold6, And Refold Methods. Proteins Struct Funct Bioinforma. 2018; 86:335-344. Doi:10.1002/Prot.25360.
MCGUFFIN LJ, Atkins J D, Salehe B R, Shuid A N, Roche D B. Intfold: An Integrated Server For Modelling Protein Structures And Functions From Amino Acid Sequences: FIG. 1. Nucleic Acids Res. 2015; 43(W1):W169-W173. Doi:10.1093/Nar/Gkv236.
BUENAVISTA MT, Roche D B, Mcguffin L J. Improvement Of 3D Protein Models Using Multiple Templates Guided By Single-Template Model Quality Assessment. Bioinformatics. 2012; 28(14):1851-1857. Doi:10.1093/Bioinformatics/Bts292.
ROCHE DB, Buenavista M T, Mcguffin L J. Funfoldqa: A Quality Assessment Tool For Protein-Ligand Binding Site Residue Predictions. Kurgan L, Ed. Plos One. 2012; 7(5):E38219. Doi:10.1371/Journal.Pone.0038219.
KÅLLBERG M, Wang H, Wang S, Et Al. Template-Based Protein Structure Modeling Using The Raptorx Web Server. Nat Protoc. 2012; 7:1511. Http://Dx.Doi.Org/10.1038/Nprot.2012.085.
MA J, Peng J, Wang S, Xu J. A Conditional Neural Fields Model For Protein Threading. Bioinformatics. 2012; 28(12):I59-I66. Doi:10.1093/Bioinformatics/Bts213.
PENG J, Xu J. A Multiple-Template Approach To Protein Threading. Proteins Struct Funct Bioinforma. 2011; 79(6):1930-1939. Doi:10.1002/Prot.23016.
PENG J, Xu J. Raptorx: Exploiting Structure Information For Protein Alignment By Statistical Inference. Proteins Struct Funct Bioinforma. 2011; 79(S10):161-171. Doi:10.1002/Prot.23175.
MA J, Wang S, Zhao F, Xu J. Protein Threading Using Context-Specific Alignment Potential. Bioinformatics. 2013; 29(13):I257-I265. Doi:10.1093/Bioinformatics/Btt210.

### REFERENCE TO A SEQUENCE LISTING

This application contains a Sequence Listing which has been submitted electronically in XML format and is hereby incorporated by reference in its entirety. The XML file was created on 2/23/26, is named CAR2.xml and is 51,326 bytes in size.

1-12. (canceled)

13. A bispecific in tandem chimeric antigen receptor (CAR) peptide, useful for inducing CAR T cell anti-tumor cell activity, said peptide comprising:
an extra cellular target antigen binding domain consisting of a single chain variable fragment (scFv) region that recognizes and ligates a surface molecule on a tumor cell,
a spacer region consisting of a IgG4-Hinge-CH₂CH₃ domain,
an IL-1 receptor type 2 domain,
a second spacer region consisting of a CD8-Hinge domain
a CD28 transmembrane motif, and
an intracellular signaling component consisting of
a 4-1BB domain and
a CD3ζ domain,
wherein the peptide, when placed in a suitable CAR T cell, will ligate to said tumor cell surface molecule inducing anti-tumor cell activity and said IL-1 receptor type 2 domain allows trapping of secreted IL-1β and thus modulation of the anti-tumor cell activity.
wherein CAR T cell action can be transiently switched off by administration of peptides that link to the IL-1 receptor type 2 domain or the scFv region.

14. The peptide as defined in claim 13 wherein said tumor cell further comprises a member of the group comprising: pancreatic cancer tumor cells, lung cancer tumor cells, ovarian cancer tumor cells and lymphoblastic leukemia tumor cells.

15. The peptide as defined in claim 13 wherein said tumor cell further comprises an acute myeloid leukemia tumor cell.

16. The peptide as defined in claim 15 wherein said acute myeloid leukemia tumor cell has a surface molecule selected from the group consisting of CD33 and CD123.

17. The peptide as defined in claim 13 wherein said IL-1 receptor type 2 is IL-1R2.

18. A method of treating cancer by CAR T cell binding to initiate killing of tumor cells comprising:
administrating to a cancer patient CAR T-cells having the peptide as defined in claim 13,
modulating anti-tumor cell activity by trapping secreted IL-1β, and
transiently switching off CAR T-cell action by administrating peptides selected from the group consisting of peptides that that link to the IL-1 receptor type 2 domain and peptides that link to the scFv region.


SEQUENCE LISTING (30 sequences)

SEQ ID NO: 1 - protein, 849 aa, synthetic construct
  REGION 1..849: note = Model 1 AntiCD33 IgG4 (HingeCH2CH3) IL1R2 (extracellular) CD8 (Hinge)
EVQLVQSGAEVKKPGSSVKVSCKASGYTITDSNIHWVRQAPGQSLEWIGYIYPYNGGTDYNQKFKNRATLTVDNPTNTAYMELSSLRSEDTAFYYCVNGNPWLAYWGQGTLVTVSSGGGGSGGGGSGGGGSDIQLTQSPSTLSASVGDRVTITCRASESLDNYGIRFLTWFQQKPGKAPKLLMYAASNQGSGVPSRFSGSGSGTEFTLTISSLQPDDFATYYCQQTKEVPWSFGQGTKVEVKRTVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKFTLQPAAHTGAARSCRFRGRHYKREFRLEGEPVALRCPQVPYWLWASVSPRINLTWHKNDSARTVPGEEETRMWAQDGALWLLPALQEDSGTYVCTTRNASYCDKMSIELRVFENTDAFLPFISYPQILTLSTSGVLVCPDLSEFTRDKTDVKIQWYKDSLLLDKDNEKFLSVRGTTHLLVHDVALEDAGYYRCVLTFAHEGQQYNITRSIELRIKKKKEETIPVIISPLKTISASLGSRLTIPCKVFLGTGTPLTTMLWWTANDTHIESAYPGGRVTEGPRQEYSENNENYIEVPLIFDPVTREDLHMDFKCVVHNTLSFQTLRTTVKETTTPAPRPPTPAPTIASQPLSLRPEACRPAAGGAVHTRGLDFACD

SEQ ID NO: 2 - protein, 632 aa, synthetic construct
  REGION 1..632: note = Model 2 AntiCD33 IgG4 (Hinge) IL1R2 (extracellular) CD8 (Hinge)
EVQLVQSGAEVKKPGSSVKVSCKASGYTITDSNIHWVRQAPGQSLEWIGYIYPYNGGTDYNQKFKNRATLTVDNPTNTAYMELSSLRSEDTAFYYCVNGNPWLAYWGQGTLVTVSSGGGGSGGGGSGGGGSDIQLTQSPSTLSASVGDRVTITCRASESLDNYGIRFLTWFQQKPGKAPKLLMYAASNQGSGVPSRFSGSGSGTEFTLTISSLQPDDFATYYCQQTKEVPWSFGQGTKVEVKRTVESKYGPPCPSCPFTLQPAAHTGAARSCRFRGRHYKREFRLEGEPVALRCPQVPYWLWASVSPRINLTWHKNDSARTVPGEEETRMWAQDGALWLLPALQEDSGTYVCTTRNASYCDKMSIELRVFENTDAFLPFISYPQILTLSTSGVLVCPDLSEFTRDKTDVKIQWYKDSLLLDKDNEKFLSVRGTTHLLVHDVALEDAGYYRCVLTFAHEGQQYNITRSIELRIKKKKEETIPVIISPLKTISASLGSRLTIPCKVFLGTGTPLTTMLWWTANDTHIESAYPGGRVTEGPRQEYSENNENYIEVPLIFDPVTREDLHMDFKCVVHNTLSFQTLRTTVKETTTPAPRPPTPAPTIASQPLSLRPEACRPAAGGAVHTRGLDFACD

SEQ ID NO: 3 - protein, 837 aa, synthetic construct
  REGION 1..837: note = Model 3 AntiCD33 IgG4 (CH2CH3) IL1R2 (extracellular) CD8 (Hinge)
EVQLVQSGAEVKKPGSSVKVSCKASGYTITDSNIHWVRQAPGQSLEWIGYIYPYNGGTDYNQKFKNRATLTVDNPTNTAYMELSSLRSEDTAFYYCVNGNPWLAYWGQGTLVTVSSGGGGSGGGGSGGGGSDIQLTQSPSTLSASVGDRVTITCRASESLDNYGIRFLTWFQQKPGKAPKLLMYAASNQGSGVPSRFSGSGSGTEFTLTISSLQPDDFATYYCQQTKEVPWSFGQGTKVEVKRTVAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKFTLQPAAHTGAARSCRFRGRHYKREFRLEGEPVALRCPQVPYWLWASVSPRINLTWHKNDSARTVPGEEETRMWAQDGALWLLPALQEDSGTYVCTTRNASYCDKMSIELRVFENTDAFLPFISYPQILTLSTSGVLVCPDLSEFTRDKTDVKIQWYKDSLLLDKDNEKFLSVRGTTHLLVHDVALEDAGYYRCVLTFAHEGQQYNITRSIELRIKKKKEETIPVIISPLKTISASLGSRLTIPCKVFLGTGTPLTTMLWWTANDTHIESAYPGGRVTEGPRQEYSENNENYIEVPLIFDPVTREDLHMDFKCVVHNTLSFQTLRTTVKETTTPAPRPPTPAPTIASQPLSLRPEACRPAAGGAVHTRGLDFACD

SEQ ID NO: 4 - protein, 804 aa, synthetic construct
  REGION 1..804: note = Model 4 Anti-CD33 IgG4 (HingeCH2CH3) IL1R2 (extracellular)
EVQLVQSGAEVKKPGSSVKVSCKASGYTITDSNIHWVRQAPGQSLEWIGYIYPYNGGTDYNQKFKNRATLTVDNPTNTAYMELSSLRSEDTAFYYCVNGNPWLAYWGQGTLVTVSSGGGGSGGGGSGGGGSDIQLTQSPSTLSASVGDRVTITCRASESLDNYGIRFLTWFQQKPGKAPKLLMYAASNQGSGVPSRFSGSGSGTEFTLTISSLQPDDFATYYCQQTKEVPWSFGQGTKVEVKRTVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKFTLQPAAHTGAARSCRFRGRHYKREFRLEGEPVALRCPQVPYWLWASVSPRINLTWHKNDSARTVPGEEETRMWAQDGALWLLPALQEDSGTYVCTTRNASYCDKMSIELRVFENTDAFLPFISYPQILTLSTSGVLVCPDLSEFTRDKTDVKIQWYKDSLLLDKDNEKFLSVRGTTHLLVHDVALEDAGYYRCVLTFAHEGQQYNITRSIELRIKKKKEETIPVIISPLKTISASLGSRLTIPCKVFLGTGTPLTTMLWWTANDTHIESAYPGGRVTEGPRQEYSENNENYIEVPLIFDPVTREDLHMDFKCVVHNTLSFQTLRTTVKE

SEQ ID NO: 5 - protein, 587 aa, synthetic construct
  REGION 1..587: note = Model 5 AntiCD33 IgG4 (Hinge) IL1R2 (extracellular)
EVQLVQSGAEVKKPGSSVKVSCKASGYTITDSNIHWVRQAPGQSLEWIGYIYPYNGGTDYNQKFKNRATLTVDNPTNTAYMELSSLRSEDTAFYYCVNGNPWLAYWGQGTLVTVSSGGGGSGGGGSGGGGSDIQLTQSPSTLSASVGDRVTITCRASESLDNYGIRFLTWFQQKPGKAPKLLMYAASNQGSGVPSRFSGSGSGTEFTLTISSLQPDDFATYYCQQTKEVPWSFGQGTKVEVKRTVESKYGPPCPSCPFTLQPAAHTGAARSCRFRGRHYKREFRLEGEPVALRCPQVPYWLWASVSPRINLTWHKNDSARTVPGEEETRMWAQDGALWLLPALQEDSGTYVCTTRNASYCDKMSIELRVFENTDAFLPFISYPQILTLSTSGVLVCPDLSEFTRDKTDVKIQWYKDSLLLDKDNEKFLSVRGTTHLLVHDVALEDAGYYRCVLTFAHEGQQYNITRSIELRIKKKKEETIPVIISPLKTISASLGSRLTIPCKVFLGTGTPLTTMLWWTANDTHIESAYPGGRVTEGPRQEYSENNENYIEVPLIFDPVTREDLHMDFKCVVHNTLSFQTLRTTVKE

SEQ ID NO: 6 - protein, 792 aa, synthetic construct
  REGION 1..792: note = Model 6 AntiCD33 IgG4 (CH2CH3) IL1R2 (extracellular)
EVQLVQSGAEVKKPGSSVKVSCKASGYTITDSNIHWVRQAPGQSLEWIGYIYPYNGGTDYNQKFKNRATLTVDNPTNTAYMELSSLRSEDTAFYYCVNGNPWLAYWGQGTLVTVSSGGGGSGGGGSGGGGSDIQLTQSPSTLSASVGDRVTITCRASESLDNYGIRFLTWFQQKPGKAPKLLMYAASNQGSGVPSRFSGSGSGTEFTLTISSLQPDDFATYYCQQTKEVPWSFGQGTKVEVKRTVAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKFTLQPAAHTGAARSCRFRGRHYKREFRLEGEPVALRCPQVPYWLWASVSPRINLTWHKNDSARTVPGEEETRMWAQDGALWLLPALQEDSGTYVCTTRNASYCDKMSIELRVFENTDAFLPFISYPQILTLSTSGVLVCPDLSEFTRDKTDVKIQWYKDSLLLDKDNEKFLSVRGTTHLLVHDVALEDAGYYRCVLTFAHEGQQYNITRSIELRIKKKKEETIPVIISPLKTISASLGSRLTIPCKVFLGTGTPLTTMLWWTANDTHIESAYPGGRVTEGPRQEYSENNENYIEVPLIFDPVTREDLHMDFKCVVHNTLSFQTLRTTVKE

SEQ ID NO: 7 - protein, 853 aa, synthetic construct
  REGION 1..853: note = Model 7 AntiCD123 IgG4 (HingeCH2CH3) IL1R2 (extracellular) CD8 (Hinge)
EVQLVQSGAEVKKPGESLKISCKGSGYSFTDYYMKWARQMPGKGLEWMGDIIPSNGATFYNQKFKGQVTISADKSISTTYLQWSSLKASDTAMYYCARSHLLRASWFAYWGQGTMVTVSSGGGGSGGGGSGGGGSDIVMTQSPDSLAVSLGERATINCESSQSLLNSGNQKNYLTWYQQKPGQPPKPLIYWASTRESGVPDRFSGSGSGTDFTLTISSLQAEDVAVYYCQNDYSYPYTFGQGTKLEIKRESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKFTLQPAAHTGAARSCRFRGRHYKREFRLEGEPVALRCPQVPYWLWASVSPRINLTWHKNDSARTVPGEEETRMWAQDGALWLLPALQEDSGTYVCTTRNASYCDKMSIELRVFENTDAFLPFISYPQILTLSTSGVLVCPDLSEFTRDKTDVKIQWYKDSLLLDKDNEKFLSVRGTTHLLVHDVALEDAGYYRCVLTFAHEGQQYNITRSIELRIKKKKEETIPVIISPLKTISASLGSRLTIPCKVFLGTGTPLTTMLWWTANDTHIESAYPGGRVTEGPRQEYSENNENYIEVPLIFDPVTREDLHMDFKCVVHNTLSFQTLRTTVKETTTPAPRPPTPAPTIASQPLSLRPEACRPAAGGAVHTRGLDFACD

SEQ ID NO: 8 - protein, 636 aa, synthetic construct
  REGION 1..636: note = Model 8 AntiCD123 IgG4 (Hinge) IL1R2 (extracellular) CD8 (Hinge)
EVQLVQSGAEVKKPGESLKISCKGSGYSFTDYYMKWARQMPGKGLEWMGDIIPSNGATFYNQKFKGQVTISADKSISTTYLQWSSLKASDTAMYYCARSHLLRASWFAYWGQGTMVTVSSGGGGSGGGGSGGGGSDIVMTQSPDSLAVSLGERATINCESSQSLLNSGNQKNYLTWYQQKPGQPPKPLIYWASTRESGVPDRFSGSGSGTDFTLTISSLQAEDVAVYYCQNDYSYPYTFGQGTKLEIKRESKYGPPCPSCPFTLQPAAHTGAARSCRFRGRHYKREFRLEGEPVALRCPQVPYWLWASVSPRINLTWHKNDSARTVPGEEETRMWAQDGALWLLPALQEDSGTYVCTTRNASYCDKMSIELRVFENTDAFLPFISYPQILTLSTSGVLVCPDLSEFTRDKTDVKIQWYKDSLLLDKDNEKFLSVRGTTHLLVHDVALEDAGYYRCVLTFAHEGQQYNITRSIELRIKKKKEETIPVIISPLKTISASLGSRLTIPCKVFLGTGTPLTTMLWWTANDTHIESAYPGGRVTEGPRQEYSENNENYIEVPLIFDPVTREDLHMDFKCVVHNTLSFQTLRTTVKETTTPAPRPPTPAPTIASQPLSLRPEACRPAAGGAVHTRGLDFACD

SEQ ID NO: 9 - protein, 841 aa, synthetic construct
  REGION 1..841: note = Model 9 AntiCD123 IgG4 (CH2CH3) IL1R2 (extracellular) CD8 (Hinge)
EVQLVQSGAEVKKPGESLKISCKGSGYSFTDYYMKWARQMPGKGLEWMGDIIPSNGATFYNQKFKGQVTISADKSISTTYLQWSSLKASDTAMYYCARSHLLRASWFAYWGQGTMVTVSSGGGGSGGGGSGGGGSDIVMTQSPDSLAVSLGERATINCESSQSLLNSGNQKNYLTWYQQKPGQPPKPLIYWASTRESGVPDRFSGSGSGTDFTLTISSLQAEDVAVYYCQNDYSYPYTFGQGTKLEIKRAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKFTLQPAAHTGAARSCRFRGRHYKREFRLEGEPVALRCPQVPYWLWASVSPRINLTWHKNDSARTVPGEEETRMWAQDGALWLLPALQEDSGTYVCTTRNASYCDKMSIELRVFENTDAFLPFISYPQILTLSTSGVLVCPDLSEFTRDKTDVKIQWYKDSLLLDKDNEKFLSVRGTTHLLVHDVALEDAGYYRCVLTFAHEGQQYNITRSIELRIKKKKEETIPVIISPLKTISASLGSRLTIPCKVFLGTGTPLTTMLWWTANDTHIESAYPGGRVTEGPRQEYSENNENYIEVPLIFDPVTREDLHMDFKCVVHNTLSFQTLRTTVKETTTPAPRPPTPAPTIASQPLSLRPEACRPAAGGAVHTRGLDFACD

SEQ ID NO: 10 - protein, 808 aa, synthetic construct
  REGION 1..808: note = Model 10 AntiCD123 IgG4 (Hinge-CH2CH3) IL1R2 (extracellular)
EVQLVQSGAEVKKPGESLKISCKGSGYSFTDYYMKWARQMPGKGLEWMGDIIPSNGATFYNQKFKGQVTISADKSISTTYLQWSSLKASDTAMYYCARSHLLRASWFAYWGQGTMVTVSSGGGGSGGGGSGGGGSDIVMTQSPDSLAVSLGERATINCESSQSLLNSGNQKNYLTWYQQKPGQPPKPLIYWASTRESGVPDRFSGSGSGTDFTLTISSLQAEDVAVYYCQNDYSYPYTFGQGTKLEIKRESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKFTLQPAAHTGAARSCRFRGRHYKREFRLEGEPVALRCPQVPYWLWASVSPRINLTWHKNDSARTVPGEEETRMWAQDGALWLLPALQEDSGTYVCTTRNASYCDKMSIELRVFENTDAFLPFISYPQILTLSTSGVLVCPDLSEFTRDKTDVKIQWYKDSLLLDKDNEKFLSVRGTTHLLVHDVALEDAGYYRCVLTFAHEGQQYNITRSIELRIKKKKEETIPVIISPLKTISASLGSRLTIPCKVFLGTGTPLTTMLWWTANDTHIESAYPGGRVTEGPRQEYSENNENYIEVPLIFDPVTREDLHMDFKCVVHNTLSFQTLRTTVKE

SEQ ID NO: 11 - protein, 591 aa, synthetic construct
  REGION 1..591: note = Model 11 AntiCD123 IgG4 (Hinge) IL1R2 (extracellular)
EVQLVQSGAEVKKPGESLKISCKGSGYSFTDYYMKWARQMPGKGLEWMGDIIPSNGATFYNQKFKGQVTISADKSISTTYLQWSSLKASDTAMYYCARSHLLRASWFAYWGQGTMVTVSSGGGGSGGGGSGGGGSDIVMTQSPDSLAVSLGERATINCESSQSLLNSGNQKNYLTWYQQKPGQPPKPLIYWASTRESGVPDRFSGSGSGTDFTLTISSLQAEDVAVYYCQNDYSYPYTFGQGTKLEIKRESKYGPPCPSCPFTLQPAAHTGAARSCRFRGRHYKREFRLEGEPVALRCPQVPYWLWASVSPRINLTWHKNDSARTVPGEEETRMWAQDGALWLLPALQEDSGTYVCTTRNASYCDKMSIELRVFENTDAFLPFISYPQILTLSTSGVLVCPDLSEFTRDKTDVKIQWYKDSLLLDKDNEKFLSVRGTTHLLVHDVALEDAGYYRCVLTFAHEGQQYNITRSIELRIKKKKEETIPVIISPLKTISASLGSRLTIPCKVFLGTGTPLTTMLWWTANDTHIESAYPGGRVTEGPRQEYSENNENYIEVPLIFDPVTREDLHMDFKCVVHNTLSFQTLRTTVKE

SEQ ID NO: 12 - protein, 796 aa, synthetic construct
  REGION 1..796: note = Model 12 AntiCD123 IgG4 (CH2CH3) IL1R2 (extracellular)
EVQLVQSGAEVKKPGESLKISCKGSGYSFTDYYMKWARQMPGKGLEWMGDIIPSNGATFYNQKFKGQVTISADKSISTTYLQWSSLKASDTAMYYCARSHLLRASWFAYWGQGTMVTVSSGGGGSGGGGSGGGGSDIVMTQSPDSLAVSLGERATINCESSQSLLNSGNQKNYLTWYQQKPGQPPKPLIYWASTRESGVPDRFSGSGSGTDFTLTISSLQAEDVAVYYCQNDYSYPYTFGQGTKLEIKRAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKFTLQPAAHTGAARSCRFRGRHYKREFRLEGEPVALRCPQVPYWLWASVSPRINLTWHKNDSARTVPGEEETRMWAQDGALWLLPALQEDSGTYVCTTRNASYCDKMSIELRVFENTDAFLPFISYPQILTLSTSGVLVCPDLSEFTRDKTDVKIQWYKDSLLLDKDNEKFLSVRGTTHLLVHDVALEDAGYYRCVLTFAHEGQQYNITRSIELRIKKKKEETIPVIISPLKTISASLGSRLTIPCKVFLGTGTPLTTMLWWTANDTHIESAYPGGRVTEGPRQEYSENNENYIEVPLIFDPVTREDLHMDFKCVVHNTLSFQTLRTTVKE

SEQ ID NO: 13 - protein, 327 aa, synthetic construct
  REGION 1..327: note = IgG4 Heavy Chain UniProtKB: P01861
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 14 - protein, 98 aa, synthetic construct
  REGION 1..98: note = CH1 domain 1 to 98
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRV

SEQ ID NO: 15 - protein, 12 aa, synthetic construct
  REGION 1..12: note = Hinge 99 to 110
ESKYGPPCPSCP

SEQ ID NO: 16 - protein, 110 aa, synthetic construct
  REGION 1..110: note = CH2 domain 111 to 220
APEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAK

SEQ ID NO: 17 - protein, 107 aa, synthetic construct
  REGION 1..107: note = CH3 domain 221 to 327
GQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 18 - protein, 398 aa, synthetic construct
  REGION 1..398: note = IL1R2 UniProtKB: P27930
MLRLYVLVMGVSAFTLQPAAHTGAARSCRFRGRHYKREFRLEGEPVALRCPQVPYWLWASVSPRINLTWHKNDSARTVPGEEETRMWAQDGALWLLPALQEDSGTYVCTTRNASYCDKMSIELRVFENTDAFLPFISYPQILTLSTSGVLVCPDLSEFTRDKTDVKIQWYKDSLLLDKDNEKFLSVRGTTHLLVHDVALEDAGYYRCVLTFAHEGQQYNITRSIELRIKKKKEETIPVIISPLKTISASLGSRLTIPCKVFLGTGTPLTTMLWWTANDTHIESAYPGGRVTEGPRQEYSENNENYIEVPLIFDPVTREDLHMDFKCVVHNTLSFQTLRTTVKEASSTFSWGIVLAPLSLAFLVLGGIWMHRRCKHRTGKADGLTVLWPHHQDFQSYPK

SEQ ID NO: 19 - protein, 13 aa, synthetic construct
  REGION 1..13: note = Signal Peptide 1 to 13
MLRLYVLVMGVSA

SEQ ID NO: 20 - protein, 330 aa, synthetic construct
  REGION 1..330: note = Extracellular Domain 14 to 343
FTLQPAAHTGAARSCRFRGRHYKREFRLEGEPVALRCPQVPYWLWASVSPRINLTWHKNDSARTVPGEEETRMWAQDGALWLLPALQEDSGTYVCTTRNASYCDKMSIELRVFENTDAFLPFISYPQILTLSTSGVLVCPDLSEFTRDKTDVKIQWYKDSLLLDKDNEKFLSVRGTTHLLVHDVALEDAGYYRCVLTFAHEGQQYNITRSIELRIKKKKEETIPVIISPLKTISASLGSRLTIPCKVFLGTGTPLTTMLWWTANDTHIESAYPGGRVTEGPRQEYSENNENYIEVPLIFDPVTREDLHMDFKCVVHNTLSFQTLRTTVKE

SEQ ID NO: 21 - protein, 26 aa, synthetic construct
  REGION 1..26: note = Transmembrane Domain 344 to 369
ASSTFSWGIVLAPLSLAFLVLGGIWM

SEQ ID NO: 22 - protein, 29 aa, synthetic construct
  REGION 1..29: note = Cytoplasmatic Domain 370 to 398
HRRCKHRTGKADGLTVLWPHHQDFQSYPK

SEQ ID NO: 23 - protein, 529 aa, synthetic construct
  REGION 1..529: note = Anti CD33 CAR 3rd Generation
EVQLVQSGAEVKKPGSSVKVSCKASGYTITDSNIHWVRQAPGQSLEWIGYIYPYNGGTDYNQKFKNRATLTVDNPTNTAYMELSSLRSEDTAFYYCVNGNPWLAYWGQGTLVTVSSGGGGSGGGGSGGGGSDIQLTQSPSTLSASVGDRVTITCRASESLDNYGIRFLTWFQQKPGKAPKLLMYAASNQGSGVPSRFSGSGSGTEFTLTISSLQPDDFATYYCQQTKEVPWSFGQGTKVEVKRTVTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRPGSRPRLEQSTSGLQNL

SEQ ID NO: 24 - protein, 245 aa, synthetic construct
  REGION 1..245: note = AntiCD33 (VH liker VL) 1 to 245
EVQLVQSGAEVKKPGSSVKVSCKASGYTITDSNIHWVRQAPGQSLEWIGYIYPYNGGTDYNQKFKNRATLTVDNPTNTAYMELSSLRSEDTAFYYCVNGNPWLAYWGQGTLVTVSSGGGGSGGGGSGGGGSDIQLTQSPSTLSASVGDRVTITCRASESLDNYGIRFLTWFQQKPGKAPKLLMYAASNQGSGVPSRFSGSGSGTEFTLTISSLQPDDFATYYCQQTKEVPWSFGQGTKVEVKRTV

SEQ ID NO: 25 - protein, 45 aa, synthetic construct
  REGION 1..45: note = CD8 Hinge 246 to 290
TTTPAPRPPTPAPTIASQPLSLRPEACRPAAGGAVHTRGLDFACD

SEQ ID NO: 26 - protein, 239 aa, synthetic construct
  REGION 1..239: note = Intracelular Domains 291 to 580
FWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRPGSRPRLEQSTSGLQNL

SEQ ID NO: 27 - protein, 516 aa, synthetic construct
  REGION 1..516: note = Anti CD123 CAR 3rd Generation
EVQLVQSGAEVKKPGESLKISCKGSGYSFTDYYMKWARQMPGKGLEWMGDIIPSNGATFYNQKFKGQVTISADKSISTTYLQWSSLKASDTAMYYCARSHLLRASWFAYWGQGTMVTVSSGGGGSGGGGSGGGGSDIVMTQSPDSLAVSLGERATINCESSQSLLNSGNQKNYLTWYQQKPGQPPKPLIYWASTRESGVPDRFSGSGSGTDFTLTISSLQAEDVAVYYCQNDYSYPYTFGQGTKLEIKRTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 28 - protein, 249 aa, synthetic construct
  REGION 1..249: note = AntiCD123 (VH liker VL) 1 to 249
EVQLVQSGAEVKKPGESLKISCKGSGYSFTDYYMKWARQMPGKGLEWMGDIIPSNGATFYNQKFKGQVTISADKSISTTYLQWSSLKASDTAMYYCARSHLLRASWFAYWGQGTMVTVSSGGGGSGGGGSGGGGSDIVMTQSPDSLAVSLGERATINCESSQSLLNSGNQKNYLTWYQQKPGQPPKPLIYWASTRESGVPDRFSGSGSGTDFTLTISSLQAEDVAVYYCQNDYSYPYTFGQGTKLEIKR

SEQ ID NO: 29 - protein, 45 aa, synthetic construct; identical to SEQ ID NO: 25
  REGION 1..45: note = CD8 Hinge 250 to 294

SEQ ID NO: 30 - protein, 222 aa, synthetic construct
  REGION 1..222: note = Intracellular Domains 295 to 517
FWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR